(19) United States
(12) Patent Application Publication
(10) Pub. No.: US 2024/0415753 A1
(43) Pub. Date: Dec. 19, 2024

(54) DEODORANT COMPOSITIONS COMPRISING CARBOXYLIC ACIDS

(71) Applicant: The Procter & Gamble Company, Cincinnati, OH (US)

(72) Inventors: Brooke Michele Cochran, Cincinnati, OH (US); Jonathan Robert Cetti, Mason, OH (US); Ryan Michael West, West Chester, OH (US); David Frederick Swaile, Cincinnati, OH (US)

(21) Appl. No.: 18/743,381

(22) Filed: Jun. 14, 2024

Related U.S. Application Data
(60) Provisional application No. 63/508,343, filed on Jun. 15, 2023.

(51) Int. Cl. A61K 8/365; A61K 8/06; A61Q 15/00; A61Q 17/00

(52) U.S. Cl. CPC A61K 8/365; A61K 8/064; A61Q 15/00; A61Q 17/005; A61K 2800/30; A61K 2800/74

(57) ABSTRACT

A deodorant composition that includes a primary carboxylic acid that has a C Log D less than −0.5 at a pH from 3.0 to 5.0. The deodorant composition is a polar in nonpolar emulsion, that includes a polar phase, a nonpolar phase and an emulsifier phase. The polar phase has a refractive index that is at most 0.01 different than the nonpolar phase. The composition is free of aluminum. The polar phase has a surface tension that is at least 20.0 mN/m greater than the nonpolar phase.

### FIELD OF THE INVENTION

The present disclosure relates to deodorant compositions and methods relating thereto.

### BACKGROUND OF THE INVENTION

One of the main functions of a deodorant or antiperspirant product is to control unpleasant body odor. At least some body odor is the result of microorganisms on the skin that break down sweat to produce the smell that is associated with body odor. Thus, there is a need for deodorant and antiperspirant compositions that neutralize body odor by preventing the bacteria that create it. Additionally, while aluminum has been used for many years as an effective odor reducer by reducing perspiration, there is consumer interest in antiperspirants and deodorants that do not contain aluminum. It can be a challenge to formulate antiperspirants and deodorants with consistent rheology and that do not result in a phase separation over time.

Thus, there is a continuing need to formulate stable aluminum-free and natural deodorants that effectively offer odor protection.

### SUMMARY OF THE INVENTION

A deodorant composition comprising: a primary carboxylic acid, wherein the primary carboxylic acid has a C Log D less than −0.5 at a pH from 3.0 to 5.0; wherein the deodorant composition is a polar in nonpolar emulsion, comprising a polar phase, a nonpolar phase and an emulsifier phase; wherein the polar phase has a refractive index that is at most 0.01 different than the nonpolar phase; wherein the composition is free of aluminum; wherein the primary carboxylic acid has a surface tension that is at least 69.8 mN/m when measured in a 2 wt % mixture in water.

A deodorant composition comprising: from about 2% to about 15% of one or more carboxylic acids comprising: one or more carboxylic acids having a C Log D of less than-0.5 at a pH from 3.0 to 5.0; and optionally one or more carboxylic acids having a C Log D of greater than −0.5 at a pH from 3.0 to 5.0; wherein the weight ratio of the C Log D greater than or equal to −0.5 at a pH from 3.0 to 5.0 to carboxylic acids having a C Log D less than-0.5 at a pH from 3.0 to 5.0 is from 0 to about 2.8:1; from about 15% to about 45% water; wherein the deodorant composition is a polar in nonpolar emulsion, comprising a polar phase, a nonpolar phase and an emulsifier phase; wherein the composition is free of aluminum.

A deodorant composition, said composition comprising: a primary carboxylic acid, wherein the primary carboxylic acid has a C Log D less than −0.5 at a pH from 3.0 to 5.0; wherein the deodorant composition is a polar in nonpolar emulsion, comprising a polar phase, a nonpolar phase and an emulsifier phase; wherein the polar phase has a refractive index that is at most 0.01 different than the nonpolar phase; wherein the composition is free of aluminum; wherein the primary carboxylic acid has a Log S greater than 2.5 mol/L at a pH from 3.0 to 5.0 at 25° C.

### BRIEF DESCRIPTION OF THE DRAWINGS

While the specification concludes with claims particularly pointing out and distinctly claiming the subject matter of the present invention, it is believed that the invention can be more readily understood from the following description taken in connection with the accompanying drawings, in which:

FIG. 1 is a graph of the C Log D of various carboxylic acids at various pH's.

FIG. 2 shows proper swabbing for the Finished Product Soleris Method.

FIG. 3 is a graph of the Log S of various carboxylic acids at various pH's.

### DETAILED DESCRIPTION OF THE INVENTION

While the specification concludes with claims that particularly point out and distinctly claim the invention, it is believed the present invention will be better understood from the following description.

The present invention can comprise, consist of, or consist essentially of the essential elements and limitations of the invention described herein, as well any of the additional or optional ingredients, components, or limitations described herein.

All percentages, parts and ratios are based upon the total weight of the compositions of the present invention, unless otherwise specified. All such weights as they pertain to listed ingredients are based on the active level and, therefore do not include carriers or by-products that may be included in commercially available materials.

The components and/or steps, including those which may optionally be added, of the various embodiments of the present invention, are described in detail below.

All documents cited are, in relevant part, incorporated herein by reference; the citation of any document is not to be construed as an admission that it is prior art with respect to the present invention.

All ratios are weight ratios unless specifically stated otherwise.

All temperatures are in degrees Celsius, unless specifically stated otherwise.

All surface tensions are in millinewtons per meter (mN/m), unless specifically stated otherwise. Newtons per meter (N/m) is the SI and IUPAC unit for measuring surface tension. For the values included here, millinewtons per meter (mN/m) is used for convenience due to the scale of the values. Millinewtons per meter (mN/m) can be converted to newtons per meter (N/m) by dividing by 1000. In some places, surface tension can also be reported in dynes per centimeter. Dynes per centimeter (dynes/cm) can be converted to millinewtons per meter (mN/m) by multiplying by 1. Dynes per centimeter (dynes/cm) and millinewtons per meter (mN/m) are equivalent units.

Except as otherwise noted, all amounts including quantities, percentages, portions, and proportions, are understood to be modified by the word “about”, and amounts are not intended to indicate significant digits.

Except as otherwise noted, the articles “a”, “an”, and “the” mean “one or more”.

Herein, “comprising” means that other steps and other ingredients which do not affect the end result can be added. This term encompasses the terms “consisting of” and “consisting essentially of”. The compositions and methods/processes can comprise, consist of, and consist essentially of the essential elements and limitations of the invention described herein, as well as any of the additional or optional ingredients, components, steps, or limitations described herein.

Herein, “effective” means an amount of a subject active high enough to provide a significant positive modification of the condition to be treated. An effective amount of the subject active will vary with the particular condition being treated, the severity of the condition, the duration of the treatment, the nature of concurrent treatment, and like factors.

The term “ambient conditions” as used herein refers to surrounding conditions under about one atmosphere of pressure, at about 50% relative humidity, and at about 25° C., unless otherwise specified. All values, amounts, and measurements described herein are obtained under ambient conditions unless otherwise specified.

As used herein, the term “carboxylic acids” includes carboxylic acids and/or salts of carboxylic acids.

The term “majority” refers to greater than about 51% of the stated component or parameter.

“Substantially free of” refers to about 2% or less, about 1% or less, or about 0.1% or less of a stated ingredient. “Free of” refers to no detectable amount of the stated ingredient or thing.

The term “volatile” as used herein refers to those materials that have a measurable vapor pressure at 25° C. Such vapor pressures typically range from about 0.01 millimeters of Mercury (mm Hg) to about 6 mmHg, more typically from about 0.02 mmHg to about 1.5 mmHg; and have an average boiling point at one (1) atmosphere of pressure of less than about 250° C., more typically less than about 235° C. Conversely, the term “non-volatile” refers to those materials that are not “volatile” as defined herein.

### Polar in Nonpolar Emulsions

A polar in nonpolar emulsion comprises, and in some cases, consists of three phases. The three phases are a polar phase, a nonpolar phase and an emulsifier phase. In a polar in nonpolar emulsion, the polar phase exists as droplets dispersed throughout the nonpolar phase. The emulsifier phase is at the interface of the polar phase and the nonpolar phase. Examples of a polar in nonpolar emulsion can be a water in oil emulsion or a water in silicone emulsion.

Examples of materials that are added to the polar phase include water, glycols, polyethers, polyglycerin, diols, carboxylic acids, sodium sulfite, sodium benzoate, organic salts and inorganic salts. Examples of materials that are added to the nonpolar phase include silicones, fragrance, triglycerides, oils and butters. One skilled in the art will know how materials are selected to be added to the polar phase and the nonpolar phase. The choice of adding materials to the polar phase or the nonpolar phase is typically determined by the solubility of a material in water. Some measures for the solubility of a material in water include Log S (pH-dependent Aqueous Solubility) and Log S0 (Intrinsic Aqueous Solubility). There may be other ways to measure the solubility of a material in water or alternatively the hydrophilicity of a material to determine if a material should be added to the polar phase or the nonpolar phase.

Log S is the pH-dependent aqueous solubility of a material. The pH-dependent aqueous solubility is defined as the equilibrium (saturated) aqueous concentration of all the solute species present in an aqueous solvent at a given pH and at 25° C. It is computed using the ACD/Labs Solubility module. For compounds with ionizable functional groups (acids and bases), the total aqueous solubility takes the apparent ionization state of each ionizable group into account through the calculation of the pKa for each functional group. The solubility is then computed at different solution pH values and reported. In this instance, the solubility of a solute is computed over the range from pH=2.0 to pH=12.0, in steps of 0.5 pH units. Log S values are expressed in units of moles per liter (mol/L) at 25° C. at the stated pH. Generally, materials with a Log S greater than about 0 mol/L at the pH of the polar phase should be added to the polar phase.

Log S0 is the intrinsic aqueous solubility of a material. Intrinsic solubility is defined as the equilibrium aqueous solubility of the neutral (un-ionized) form of a molecule in an aqueous solvent in the absence of acid or alkali at 25° C. It is computed in this instance using the ACD/Labs Solubility module and the ACD/Labs GALAS algorithm. For a non-ionizable and neutral compound, the total aqueous solubility equals the intrinsic aqueous solubility because only the neutral species is involved. For a compound with ionizable groups, the solubility expression is more complex because multiple species (ionization states) with varying solubility are present, depending on the pH of the solution, and Log S should be used instead. Log S0 values are expressed in units of mol/L at 25° C. Generally, materials with a Log S0 greater than about 0 mol/L should be added to the polar phase.

The emulsifier phase constitutes the one or more emulsifiers that are used to separate the polar and nonpolar phases. An emulsifier will have a HLB. The HLB scale ranges from 0 to 20, with 10 corresponding to an emulsifier that is equally attracted to water and oil. Emulsifiers with HLB values less than 10 are more hydrophobic and thus better at stabilizing polar in nonpolar emulsions. Example suitable emulsifiers are surfactants, polymeric emulsifiers, silicone RAKE emulsifiers, polyether modified silicones, polyglycerin modified silicones and emulsifying elastomers. Some suitable emulsifiers include PEG/PPG-18/18 Dimethicone, PEG/PPG-20/15 Dimethicone, Sorbitan Laurate (Span™ 20), Sorbitan Palmitate (Span™ 40), Sorbitan Stearate (Span™ 60), Sorbitan Oleate (Span™ 80), Sorbitan Trioleate (Span™ 85), Polyglyceryl-2 Dipolyhydroxystearate, Glyceryl Oleate, Methyl Glucose Sesquistearate, Methyl Glucose Dioleate, Lauryl PEG/PPG-18/18 Methicone, PEG-10 Dimethicone, PEG/PPG-19/19 Dimethicone, Lauryl PEG-10 Tris(trimethylsiloxy) silyethyl Dimethicone, Cetyl Diglycerl Tris(trimethylsiloxy) silyethyl Dimethicone, PEG-9 Polydimethylsiloxyethyl Dimethicone, Cetyl PEG/PPG/10/1 Dimethicone, Lauryl Polyglyceryl-3 Polydimethylsiloxyethyl Dimethicone, Dimethicone Crosspolymers and similar materials. Emulsifiers can be supplied at 100 wt % or can be supplied dispersed in a silicone or nonpolar oil. When the emulsifier is supplied dispersed in a silicone or a nonpolar oil, the emulsifier will be in the emulsifier phase and the silicone or nonpolar oil that it is dispersed in will be in the nonpolar phase.

A high internal phase polar in nonpolar emulsion may be used to create a solid-like gel that is preferred by some consumers. A high internal phase polar in nonpolar emulsion means that the weight fraction of the polar phase is higher than the weight fraction of the nonpolar phase. It is preferred for the polar phase to comprise at least about 75% of the composition, or alternatively at least about 80% of the composition, or alternatively at least about 82% of the composition, or alternatively at least about 83% of the composition.

### Carboxylic Acids

Carboxylic acids are a class of materials that have been used in the cosmetic industry and are effective in providing anti-aging and moisture retention benefits to the skin. Some carboxylic acids are known to be effective antimicrobials. Carboxylic acids can be linear or branched, saturated or unsaturated, or contain additional hydroxy groups beyond the carboxylic acid moiety. Monocarboxylic acids can be defined as having a single carboxylic acid moiety with any of the preceding characteristics. Dicarboxylic acids can be defined as having two carboxylic acid moieties on a molecule with any of the preceding characteristics.

Carboxylic acids can be used to prevent odor-causing bacteria by buffering the underarm skin at a lower pH. Carboxylic acids can be used as deodorant actives. Deodorant actives can include any topical material that is known or otherwise effective in preventing or eliminating malodor associated with perspiration. However, the use of certain carboxylic acids in polar in nonpolar emulsions can decrease the rheology of the emulsion over time and can lead to phase separation. Loss of rheology over time is an indication that emulsion droplets are coalescing. As coalescence progresses, the emulsion will fully split into a nonpolar phase on top of a polar phase due to the density difference between the phases. Accelerated decreasing of emulsion rheology is an indication that phase separation will also occur more readily. The present inventors have found that a consistent emulsion rheology over time with no phase separation of a polar in nonpolar emulsion comprising a carboxylic acid can be achieved by using only acids that maintain a sufficiently higher surface tension in the polar phase than the nonpolar phase. The consistent rheology over time can be determined by evaluating the percent change in the complex modulus over time, according to the Complex Modulus Rheology Method Test Method, described hereafter. Compositions were determined to have no phase separation if no phase separation was visually discernible. As used herein, “visually discernible” means that a human viewer can see if the product had separated into 2 distinct layers with the unaided eye (except for standard corrective lenses adapted to compensate for near-sightedness, farsightedness, or astigmatism, or other corrected vision) in lighting at least equal to the illumination of a standard 100-watt incandescent white light bulb at 30 cm.

It was surprisingly found that even a small increase in polar phase surface tension significantly improved rheology consistency over time. The present inventors have additionally found that the impact of a carboxylic acid on the surface tension of the polar phase, and therefore the surface tension difference between the polar and nonpolar phases, can be determined by measuring the surface tension of 2 wt % solution of the carboxylic acid in water. Table 1 below shows the surface tension of various carboxylic acids at a 2 wt % solution in water. Surface tension was measured using surface tension measurement, described herein. Measurement time period was 1 minute with 30 values measured over the measurement time period. Surface tension of a sample is calculated as the average of the last 5 measured values. Each reported value is an average of 3 samples measured. It is preferred if the surface tension of the acid when measured at 2 wt % in water is at least 69.8 mN/m, or at least 67 mN/m, or at least 65 mN/m, or at least 64 mN/m. The surface tension of the primary carboxylic acid when measured at 2 wt % water can be greater than or equal to 64 mN/m, alternatively greater than or equal to 66 mN/m, alternatively greater than or equal to 68 mN/m, and alternatively greater than or equal to 70 mN/m. The surface tension of the primary carboxylic acid when measured at 2 wt % water can be less than 100 mN/m, alternatively less than 85 mN/m, alternatively less than 80 mN/m, alternatively less than 78 mN/m, and alternatively less than 75 mN/m.

The weight ratio of carboxylic acid having a surface tension that is less than 69.8 mN/m when measured in a 2 wt % mixture in water to carboxylic acid having a surface tension that is at least 69.8 mN/m when measured in a 2 wt % mixture in water can be from 0 to about 2.8:1, alternatively 0 to about 2.5:1, alternatively 0 to about 2:1, alternatively 0 to about 1.5:1, alternatively 0 to about 1:1, alternatively 0 to about 0.75:1, alternatively 0 to about 0.5:1, alternatively 0 to about 0.25:1, alternatively 0 to about 0.1:1, alternatively from 0 to about 0.05:1. The composition can be substantially free of and/or free of carboxylic acid less than 69.8 mN/m when measured in a 2 wt % mixture in water and/or carboxylic acid less than 69.8 mN/m when measured in a 2 wt % mixture in water are not intentionally added.

TABLE 1

| | Surface Tension |
|---|---|
| | when measured at |
| | 2 wt % in Water |
| Malic Acid | 71.8 mN/m |
| Glycolic Acid | 71.1 mN/m |
| Citric Acid | 71.3 mN/m |
| Succinic Acid | 69.8 mN/m |
| Ascorbic Acid | 72.4 mN/m |
| Mandelic Acid | 63.7 mN/m |
| Lactic Acid | 63.7 mN/m |

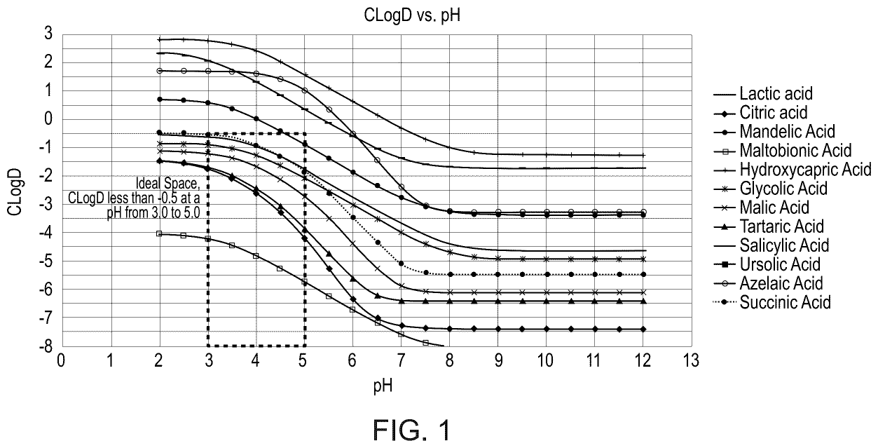

The present inventors have additionally found that the hydrophilicity of a carboxylic acid, specifically the C Log D of the material, impacts rheology of the emulsion over time. C log D is the pH-dependent octanol: water partition coefficient for ionizable compounds. The pH-Dependent Octanol-Water Partition Coefficient (log D) is defined as the equilibrium distribution between a non-polar octanol phase and a polar aqueous phase of all solute species present at a given pH (of the aqueous phase) at 25° C. It is computed in this instance using the ACD/Labs Log D module. For compounds with ionizable functional groups (acids and bases), the partition coefficient calculation takes the apparent ionization state of each ionizable group into account through the calculation of the pKa for each functional group. The partition coefficient is then computed at different solution pH values and reported. In this instance, the log D of a solute is computed over the range from pH=2.0 to pH=12.0, in steps of 0.5 pH units. If the C Log D of the carboxylic acid is too high, the carboxylic acid will not be sufficiently soluble in the polar phase. When this occurs, the acid will have a tendency to migrate towards the emulsion interface and disrupt the emulsification properties of the emulsifier used to separate the polar and nonpolar phases. It is preferred that a carboxylic acid has C Log D less than −0.5 at a pH from 3.0 to 5.0. It is also preferred that a carboxylic acid has a C Log D less than −0.5 at a pH from 3.0 to 4.5. To be preferred over a pH range means than the C Log D can be less than −0.5 for over 50% of the pH range. FIG. 1 is a graph of the C Log D of various carboxylic acids at various pH's.

The weight ratio of carboxylic acid having a C Log D greater than or equal to −0.5 at a pH from 3.0 to 5.0 to carboxylic acid has C Log D less than −0.5 at a pH from 3.0 to 5.0 can be from 0 to about 2.8:1, alternatively 0 to about 2.5:1, alternatively 0 to about 2:1, alternatively 0 to about 1.5:1, alternatively 0 to about 1:1, alternatively 0 to about 0.75:1, alternatively 0 to about 0.5:1, alternatively 0 to about 0.25:1, alternatively 0 to about 0.1:1, alternatively from 0 to about 0.05:1. The composition can be substantially free of and/or free of carboxylic acid having a C Log D greater than or equal to −0.5 at a pH from 3.0 to 5.0 and/or carboxylic acid having a C Log D greater than or equal to −0.5 at a pH from 3.0 to 5.0 are not intentionally added.

The composition can be substantially free of or free of mandelic acid and/or salicylic acid.

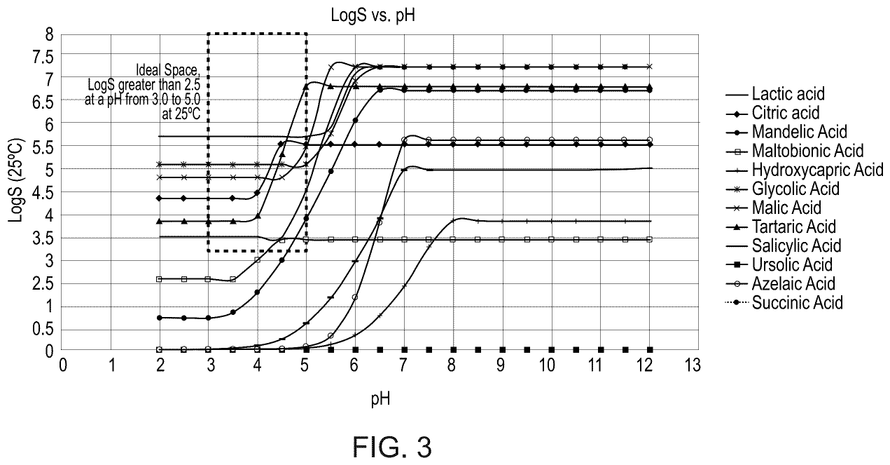

The present inventors have additionally found that the water solubility of a carboxylic acid, specifically the Log S of the material, impacts rheology of the emulsion over time. If the Log S of the carboxylic acid is too low, the carboxylic acid will not be sufficiently soluble in the polar phase. When this occurs, the acid will have a tendency to migrate towards the emulsion interface and disrupt the emulsification properties of the emulsifier used to separate the polar and nonpolar phases. It is preferred that a carboxylic acid has Log S greater than 2.5 mol/L at a pH from 3.0 to 5.0. It is also preferred that a carboxylic acid has a Log S greater than 2.5 mol/L at a pH from 3.0 to 4.5. To be preferred over a pH range means that the Log S can be greater than 2.5 mol/L for over 50% of the pH range. FIG. 3 is a graph of the Log S of various carboxylic acids at various pH's.

The weight ratio of carboxylic acid having a Log S less than or equal to 2.5 at a pH from 3.0 to 5.0 at 25° C. to carboxylic acid has Log S greater than 2.5 at a pH from 3.0 to 5.0 at 25° C. can be from 0 to about 2.8:1, alternatively 0 to about 2.5:1, alternatively 0 to about 2:1, alternatively 0 to about 1.5:1, alternatively 0 to about 1:1, alternatively 0 to about 0.75:1, alternatively 0 to about 0.5:1, alternatively 0 to about 0.25:1, alternatively 0 to about 0.1:1, alternatively from 0 to about 0.05:1. The composition can be substantially free of and/or free of carboxylic acid having a Log S greater than 2.5 at a pH from 3.0 to 5.0 at 25° C. and/or carboxylic acid having a Log S greater than 2.5 at a pH from 3.0 to 5.0 at 25° C. from 3.0 to 5.0 are not intentionally added.

Primary carboxylic acid refers to the carboxylic acid and/or salts thereof used at the highest concentration in the formula by weight percentage. If two or more carboxylic acids and/or salts thereof are used at equal concentration than the two or more carboxylic acids are considered primary, and the weight % is the sum of the two or more carboxylic acids. Additional carboxylic acids and/or salts thereof may be included as secondary, tertiary, etc. carboxylic acids. Preferred primary carboxylic acids can include malic acid, glycolic acid, citric acid, succinic acid, salts thereof or mixtures thereof and salts can include potassium and/or sodium salts. The carboxylic acid can include lactic acid, malic acid, tartaric acid, succinic acid, vitamin C, tetrahydrofuran dicarboxylic acid, glycolic acid, oxalic acid, gluconic acid, and salts thereof and mixtures thereof. The amount of primary carboxylic acid may be at least about 0.5%, at least about 1%, at least about 1.5%, at least about 2%, at least about 3%, or at least about 3.5%. The amount of primary carboxylic acid may range from about 0.5% to about 15%, alternatively from about 0.5% to about 13%, alternatively from about 0.5% to about 10%, alternatively from about 0.5% to about 8%, alternatively from about 0.5% to about 6%, alternatively from about 1% to about 5%, or alternatively from about 1.5% to about 4%, by weight of the composition. The amount of primary carboxylic acid may range from about 2% to about 15%, alternatively from about 2.5% to about 12%, alternatively from about 3% to about 10%, alternatively from about 3.5% to about 9%, and alternatively from about 4% to 8%. The composition may have only one carboxylic acid, but when there is more than one carboxylic acid, the total amount of carboxylic acids, by weight of the composition, may be the same as the amounts and ranges described above.

Stability of a formulation can be tested at an elevated temperature. Elevated temperatures increase the kinetics within a mixture. Elevated temperatures increase the collision rate of droplets within an emulsion, providing more frequent opportunities for the droplets to collide and coalesce. For example, 49° C. can be used to accelerate the stability assessment of an emulsion. The emulsion can be placed at 49° C. for a period of 1, 2, 3, 4, 5, or 6 weeks. For example, 40° C. can also used to accelerate the stability assessment of an emulsion. The emulsion can be placed at 40° C. for a period of 1, 2, or 3 months. Because elevated temperatures are used to assess stability, surface tension of the polar and nonpolar phases may also be measured at an elevated temperature of for example 49° C. or 40° C.

In addition, carboxylic acids may be used in deodorant compositions that are desired to be clear. For the composition to appear clear, the refractive index of the polar and nonpolar phases may not vary too much. For example, for a clear appearance of a polar in nonpolar emulsion, a significant amount of material with high refractive index, water soluble solvents or emollients (for example glycols, polyethers, diols, glycerin, or mixtures thereof), can be added to the polar phase to match the refractive index of water (1.333) to silicone (1.40). However, polar materials that can raise the refractive index have also been found to decrease rheology over time and can lead to phase separation because these materials also decrease the surface tension of the polar phase. In a clear polar in nonpolar emulsion, the choice of carboxylic acid is further constrained to maintain sufficient surface tension difference between the polar and nonpolar phases. Table 2 below shows how different solvents impact the surface tension of water. Surface tension is measured for mixtures of solvents at 50 wt % in water. Surface tension was measured using the surface tension measurement, as described in the Test Method section herein. Measurement time period was 1 minute with 30 values measured over the measurement time period. Surface tension of a sample is calculated as the average of the last 5 measured values. Each reported value is an average of 3 samples measured. Based on this data, glycerin may be preferred over PEGs, PEGs are preferred over PG, and PG is preferred over DPG to maximize the surface tension difference between the polar and nonpolar phases.

TABLE 2

| | Surface Tension |
|---|---|
| | when measured at |
| | 50 wt % in Water |
| Glycerin | 68.6 mN/m |
| PEG-8 (PEG 400) | 51.8 mN/m |
| PEG-12 (PEG 600) | 49.6 mN/m |
| Propylene Glycol (PG) | 44.6 mN/m |
| Dipropylene Glycol (DPG) | 38.9 mN/m |

This mode of action for prevention of bacterial growth by carboxylic acids may also be pH dependent, thus the formulation pH is important. Maintaining a pH near the pKa or lower will keep the acid protonated, which improves ingredient potential for control of bacteria. For a consumer-preferred feel on the skin, there is also a lower limit for the pH to stay above. Formulations may balance these two factors.

Also important to an effective formulation may be the inclusion of an additional antimicrobial. The inclusion of certain carboxylic acids alone provides good prevention of bacterial growth, but in order to achieve an even higher level of bacterial growth inhibition and corresponding odor protection, an additional antimicrobial may be used. For example, additional antimicrobials may include, without being limited to, hexamidine, thymol, polyvinyl formate, niacinamide, cinnamon essential oil, cinnamon bark essential oil, cinnamic aldehyde, piroctone olamine, octenidine dihydrochloride, polydialyldimethylammonium chloride, polyquaternium, and combinations thereof. The antimicrobial may be a quaternary antimicrobial such as octenidine dihydrochorloide or polyquaternium.

The inventors believe that octenidine dihydrochloride or piroctone olamine may be good antimicrobials to combine with carboxylic acids. Without being bound by theory, the present inventors believe that the carboxylic acids of the claimed invention can disrupt the lipophilic wall of the cell membrane, thus enabling the antimicrobial to penetrate the cell wall more effectively and lessen the propagation of bacteria growth. While previous studies have shown that quaternary antimicrobials will have a higher degree of hostility at an alkaline pH (See “pH Influence on Antibacterial Efficacy of Common Antiseptic Substances”, by Cornelia Wiegand, Martin Abel, Peter Ruth, Peter Elsner, and Uta-Christina Hipler, published Jan. 20, 2015 in Skin Pharmacology and Physiology, Skin Pharmacol Physiol 2015; 28:147-158), the present inventors have unexpectedly discovered that some antimicrobials can contribute to a higher degree of hostility at a lower pH when combined with the carboxylic acids of the claimed invention. (Sec tables 6 and 7 for further discussion).

### Glycols and Polyether Compounds

Deodorant formulations may optionally contain glycols. When used as a carrier, glycols are known in the art to promote a hostile environment for bacterial growth. Glycol materials may include, but are not limited to, dipropylene glycol, propylene glycol, 1,3 Propanediol, butylene glycol, tripropylene glycol, hexylene glycol, 1,2 hexane diol, PPG-10 butanediol, and polyethylene glycol. Glycols may be particularly useful for aqueous composition, to provide solvency for lipophilic materials such as carboxylic acids and antimicrobials. For compositions containing silicone or triglycerides as a carrier, glycols may not be needed to solubilize carboxylic acids and antimicrobials. In such compositions, glycols may disrupt the creation of a solid stick by inhibiting the proper crystallization of the wax structurants due to the high degree of polarity coming from the short chain glycols, thus not achieving the desired hardness.

Dipropylene glycol and propylene glycol are both known in the art to promote a hostile environment to bacterial growth. The inventors believe that dipropylene glycol is a good carrier to combine with other antimicrobials. Deodorant compositions may comprise from about 0% to about 65%, from about 0% to about 50%, from about 10% to about 55%, from about 10% to about 50%, from about 20% to about 50%, from about 30% to about 50%, or from about 30% to about 55%, of any glycol disclosed herein, but dipropylene glycol or propylene glycol in particular, by weight of the composition. The glycols may be a single or a combination of short-chain glycols, or a combination of short-chain and longer-chain glycols. A short-chain glycol means comprising at most a C12 chain length, alternatively at most a C11, C10, or C9 chain length.

Glycols may be used in clear gel deodorant compositions that may be water-in-oil emulsions. The glycols may be used to adjust the refractive index of the water phase so that it matches the refractive index of the oil phase (preferably to within about 0.0004) in order to achieve maximum clarity of the final composition. For optimum clarity the refractive index of the oil phase and the water phase should be matched to within about 0.001 or better, alternatively to within about 0.0004.

Deodorant formulations may optionally contain a polyether compound. Polyether compounds may include, but are not limited to, polyethylene glycols and polypropylene glycols. Polyether compounds suitable for use in the deodorant compositions include, but are not limited to, PEG-4 (also called PEG 200 or polyethylene glycol with average molecular weight of 200 daltons), PEG-6 (also called PEG 300 or polyethylene glycol with average molecular weight of 300 daltons), PEG-8 (also called PEG 400 or polyethylene glycol with average molecular weight of 400 daltons), PEG-12 (also called PEG 600 or polyethylene glycol with average molecular weight of 600 daltons), polypropylene glycol (like dipropylene glycol, tripropylene glycol, PPG-3, PPG-6, PPG-9, PPG-12, PPG-15, etc.), diethylene glycol, triethylene glycol, and combinations thereof.

Polyether compounds may be particularly useful for aqueous compositions to provide solvency for lipophilic materials such as antimicrobials. For clear gel deodorant compositions, polyether compounds are particularly useful as these materials have a higher refractive index than glycols and therefore are more effective at adjusting the refractive index of the water (also referred to as polar or aqueous) phase to the oil (also referred to as nonpolar or silicone) phase in order to achieve maximum clarity of the final composition. Polyether compounds may be preferred to glycols due to higher polar phase surface tension. Deodorant compositions may comprise from about 0% to about 65%, from about 0% to about 50%, from about 10% to about 55%, from about 10% to about 50%, from about 20% to about 50%, from about 30% to about 50%, from about 30% to about 55%, from about 20% to about 45%, or from about 25% to about 40% of any polyether compound disclosed herein by weight of the composition.

The gel composition may have a clarity better than 100 NTU (Nephelometric Turbidity Units), preferably better than 75 NTU, and most preferably better than 50 NTU at 21° C.

There may be a water phase and a silicone phase. The refractive index of the water phase may be the same as the silicone phase, to within about 0.01, to within about 0.008, to within about 0.004, to within about 0.001 or better, preferably to within about 0.0004.

The composition may have a percent transmittance (% T) of at least about 80% transmittance at 600 nm. In some embodiments, the refractive index of the water phase may be from 1.3500 to 1.4300.

The composition may comprise at least 3% ethanol, by weight of the composition, or at least 0.25% of an ionic salt, by weight of the composition, and the ionic salt may be sodium chloride.

### Deodorant Compositions

The deodorant compositions may take one of many forms. Inventive forms may include a roll-on, solid sticks, (clear) gels, soft solid, sprays, cream, lotion, or serum. Below are lists of materials for various forms of the deodorant compositions. A roll-on deodorant composition can comprise, for example, water, emollient, solubilizer, deodorant actives, antioxidants, preservatives, or combinations thereof. A clear gel deodorant composition can comprise, for example, water, emollient, solubilizer, deodorant actives, antioxidants, preservatives, ethanol, or combinations thereof. A solid stick deodorant composition can comprise, for example emollient, deodorant actives, waxes, or combinations thereof.

### Water

The deodorant composition can include water. Water can be present in an amount of about 1% to about 99.5%, about 1% to about 30%, about 1% to about 50%, about 10% to about 45%, from about 15% to about 45%, from about 20% to about 45%, from about 30% to about 45%. Water can be present in an amount of about 25% to about 99.5%, about 50% to about 95%, about 50% to about 99.5%, about 75% to about 99.5% about 80% to about 99.5%, or any combination of the end points and points encompassed within the ranges, by weight of the deodorant composition.

### Deodorant Actives

Suitable deodorant actives can include any topical material that is known or otherwise effective in preventing or eliminating malodor associated with perspiration. Suitable deodorant actives may be selected from the group consisting of antimicrobial agents (e.g., bacteriocides, fungicides), malodor-absorbing material, and combinations thereof. For example, antimicrobial agents may comprise cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, sodium aluminum chlorohydroxy lactate, triethyl citrate, tricetylmethyl ammonium chloride, 2,4,4′-trichloro-2′-hydroxy diphenyl ether (triclosan), 3,4,4′-trichlorocarbanilide (triclocarban), diaminoalkyl amides such as L-lysine hexadecyl amide, heavy metal salts of citrate, salicylate, and piroctose, especially zinc salts, and acids thereof, heavy metal salts of pyrithione, especially zinc pyrithione, zinc phenolsulfate, farnesol, and combinations thereof. The concentration of the optional deodorant active may range from about 0.001%, from about 0.01%, of from about 0.1%, by weight of the composition to about 20%, to about 10%, to about 5%, or to about 1%, by weight of the composition.

### Odor Entrappers

The composition can include an odor entrapper. Suitable odor entrappers for use herein include, for example, solubilized, water-soluble, uncomplexed cyclodextrin. As used herein, the term “cyclodextrin” includes any of the known cyclodextrins such as unsubstituted cyclodextrins containing from six to twelve glucose units, especially, alpha-cyclodextrin, beta-cyclodextrin, gamma-cyclodextrin and/or their derivatives and/or mixtures thereof. The alpha-cyclodextrin consists of six glucose units, the beta-cyclodextrin consists of seven glucose units, and the gamma-cyclodextrin consists of eight glucose units arranged in a donut-shaped ring. The specific coupling and conformation of the glucose units give the cyclodextrins a rigid, conical molecular structure with a hollow interior of a specific volume. The “lining” of the internal cavity is formed by hydrogen atoms and glycosidic bridging oxygen atoms, therefore this surface is fairly hydrophobic. The unique shape and physical-chemical property of the cavity enable the cyclodextrin molecules to absorb (form inclusion complexes with) organic molecules or parts of organic molecules which can fit into the cavity. Many perfume molecules can fit into the cavity.

Cyclodextrin molecules are described in U.S. Pat. Nos. 5,714,137, and 5,942,217. Suitable levels of cyclodextrin are from about 0.1% to about 5%, alternatively from about 0.2% to about 4%, alternatively from about 0.3% to about 3%, alternatively from about 0.4% to about 2%, by weight of the composition.

### Buffering Agent

The composition can include a buffering agent which may be alkaline, acidic or neutral. The buffer can be used in the composition for maintaining the desired pH. The composition may have a pH from about 3.25 to about 6, from about 3.5 to about 5.5, or from about 3.7 to about 5.

Suitable buffering agents include, for example, hydrochloric acid, sodium hydroxide, potassium hydroxide, and combinations thereof.

The compositions can contain at least about 0%, alternatively at least about 0.001%, alternatively at least about 0.01%, by weight of the composition, of a buffering agent. The composition may also contain no more than about 1%, alternatively no more than about 0.75%, alternatively no more than about 0.5%, by weight of the composition, of a buffering agent.

The deodorant compositions and/or the polar phase may have a pH of at least about 3.25, alternatively at least about 3.5, alternatively at least about 3.7, according to the pH Test Method, described hereafter. The deodorant and/or the polar phase may have a pH of at least about 3.5, or at least about 3.7, according to the pH Test Method, described hereafter.

### Chelator

The deodorant compositions may comprise a chelator. Specific and/or additional chelators may include, but are not limited to, diethylenetriaminepentaacetic acid (DTPA), diethylenetriaminepentakis (methylenephosphonic acid) (DTPMP), desferrioxamine, their salts and combinations thereof, EDTA, DPTA, EDDS, enterobactin, desferrioxamine, HBED, and combinations thereof. The amount of chelant, by weight of composition, may be from about 0.05% to about 4%.

### Solubilizer or Emulsifiers

The composition can contain a solubilizer or emulsifier. A suitable solubilizer or emulsifier can be, for example, a surfactant, such as a no-foaming or low-foaming surfactant. Suitable surfactants are nonionic surfactants, cationic surfactants, amphoteric surfactants, zwitterionic surfactants, and mixtures thereof.

Suitable solubilizers and emulsifiers include, for example, hydrogenated castor oil, polyoxyethylene 2 stearyl ether, polyoxyethylene 20 stearyl ether, PEG/PPG-18/18 Dimethicone and combinations thereof. One suitable emulsifier that may be used in the present composition is PEG/PPG-18/18 Dimethicone.

When the solubilizing agent is present, it is typically present at a level of from about 0.01% to about 15%, alternatively from about 0.01% to about 10%, alternatively from about 0.05% to about 5%, alternatively from about 0.01% to about 3%, by weight of the composition.

### Preservatives

The composition can include a preservative. The preservative is included in an amount sufficient to prevent spoilage or prevent growth of inadvertently added microorganisms for a specific period of time, but not sufficient enough to contribute to the odor neutralizing performance of the composition. In other words, the preservative is not being used as the antimicrobial compound to kill microorganisms on the surface onto which the composition is deposited in order to eliminate odors produced by microorganisms. Instead, it is being used to prevent spoilage of the composition in order to increase shelf-life.

The preservative can be any organic preservative material which will not cause damage to fabric appearance, e.g., discoloration, coloration, bleaching. Suitable water-soluble preservatives include organic sulfur compounds, halogenated compounds, cyclic organic nitrogen compounds, low molecular weight aldehydes, parabens, propane diaol materials, isothiazolinones, quaternary compounds, benzoates, low molecular weight alcohols, dehydroacetic acid, phenyl and phenoxy compounds, sodium benzoate, sodium salicylate, or mixtures thereof.

Non-limiting examples of commercially available water-soluble preservatives include a mixture of about 77% 5-chloro-2-methyl-4-isothiazolin-3-one and about 23% 2-methyl-4-isothiazolin-3-one, a broad spectrum preservative available as a 1.5% aqueous solution under the trade name Kathon® CG by Rohm and Haas Co.; 5-bromo-5-nitro-1,3-dioxane, available under the tradename Bronidox L® from Henkel; 2-bromo-2-nitropropane-1,3-diol, available under the trade name Bronopol® from Inolex; 1,1′-hexamethylene bis(5-(p-chlorophenyl) biguanide), commonly known as chlorhexidine, and its salts, e.g., with acetic and digluconic acids; a 95:5 mixture of 1,3-bis(hydroxymethyl)-5,5-dimethyl-2,4-imidazolidinedione and 3-butyl-2-iodopropynyl carbamate, available under the trade name Glydant Plus® from Lonza; N-[1,3-bis(hydroxymethyl) 2,5-dioxo-4-imidazolidinyl]-N,N′-bis(hydroxy-methyl) urea, commonly known as diazolidinyl urea, available under the trade name Germall® II from Sutton Laboratories, Inc.; N,N″-methylenebis {N′-[1-(hydroxymethyl)-2,5-dioxo-4-imidazolidinyl]urea}, commonly known as imidazolidinyl urea, available, e.g., under the trade name Abiol® from 3V-Sigma, Unicide U-13® from Induchem, Germall 115® from Sutton Laboratories, Inc.; polymethoxy bicyclic oxazolidine, available under the trade name Nuosept® C from Hüls America; formal-dehyde; glutaraldehyde; polyaminopropyl biguanide, available under the trade name Cosmocil CQ® from ICI Americas, Inc., or under the trade name Mikrokill® from Brooks, Inc; dehydroacetic acid; and benzsiothiazolinone available under the trade name Koralone™ B-119 from Rohm and Hass Corporation.

Suitable levels of preservative can range from about 0.0001% to about 0.5%, alternatively from about 0.0002% to about 0.2%, alternatively from about 0.0003% to about 0.1%, by weight of the composition.

### Emollient

The deodorant composition can comprise an emollient system including at least one emollient, but it could also be a combination of emollients. Suitable emollients are often liquid under ambient conditions. Depending on the type of product form desired, concentrations of the emollient(s) in the deodorant compositions can range from about 1% to about 95%, from about 5% to about 95%, from about 15% to about 75%, from about 1% to about 10%, from about 15% to about 45%, or from about 1% to about 30%, by weight of the deodorant composition.

Emollients suitable for use in the deodorant compositions include, but are not limited to, propylene glycol, polypropylene glycol (like dipropylene glycol, tripropylene glycol, etc.), diethylene glycol, triethylene glycol, PEG-4, PEG-8, 1,3 propanediol, 1,2 pentanediol, 1,2 hexanediol, hexylene glycol, glycerin, C2 to C20 monohydric alcohols, C2 to C40 dihydric or polyhydric alcohols, alkyl ethers of polyhydric and monohydric alcohols, volatile silicone emollients such as cyclopentasiloxane, nonvolatile silicone emollients such as dimethicone, mineral oils, polydecenes, petrolatum, and combinations thereof. One example of a suitable emollient comprises PPG-15 stearyl ether. Other examples of suitable emollients include dipropylene glycol and propylene glycol.

Emollients may include, but are not limited to, certain liquid triglycerides, certain monoalkylglycol dialkyl acid esters and silicones.

As consumers seek more natural ingredients in their deodorants, one approach to formulation is to use emollients derived from natural oils. Emollients derived from natural oils are derived from plant sources, such as palm oil or coconut oil. The emollient derived from natural oils may be a liquid triglyceride, defined as liquid at 25° C. Thus, products that hope to emphasize natural ingredients may have a significant amount of a liquid triglyceride, for example. Derived directly from plant sources, liquid triglycerides are often short chains. Longer chain triglycerides may be used as structurants in deodorant or antiperspirant sticks, but the triglycerides of the compositions described herein are generally liquid at room temperature (25° C.) and tend to be shorter chains (e.g., from C8 to C10). The liquid triglyceride may be caprylic/capric triglyceride (coconut oil fractionated) or triheptanoin.

The deodorant sticks may comprise at least about 25% of one or more liquid triglyceride, alternatively at least about 30%, at least 35%, alternatively at least about 40%, alternatively at least about 45%, or alternatively at least about 50% liquid triglyceride, by weight of the composition. The deodorant stick may comprise from about 25% to about 60%, by weight of the composition, of one or more liquid triglyceride, alternatively from about 25% to about 50%, alternatively from about 30% to about 50%, alternatively from about 35% to about 60%, alternatively from about 35% to about 50%, alternatively from about 40% to about 60%, or alternatively from about 40% to about 50%, by weight of the composition, of one or more liquid triglyceride. In general, the greater amount of liquid in the formulation, the softer the deodorant stick may be. The more solids in the formulation leads to greater hardness. Because achieving a sufficient softness in a deodorant stick with natural ingredients can be a challenge, it can be beneficial to formulate with higher amounts of liquids such as liquid triglyceride. The level of liquid triglyceride as referred to herein may be the sum total of one or more types of liquid triglyceride in a particular deodorant stick.

Additional emollients may be used, such as plant oils (generally used at less than 10%) including olive oil, coconut oil, sunflower seed oil, jojoba seed oil, avocado oil, canola oil, and corn oil. Additional emollients including mineral oil; shea butter, PPG-14 butyl ether; isopropyl myristate; petrolatum; butyl stearate; cetyl octanoate; butyl myristate; myristyl myristate; C12-15 alkylbenzoate (e.g., Finsolv™); octyldodecanol; isostearyl isostearate; octododecyl benzoate; isostearyl lactate; isostearyl palmitate; isobutyl stearate; carbonates (e.g., dipropylheptyl carbonate, dicaprylyl carbonate); ethers (e.g., dicaprylyl ether); esters (e.g., ethyl macadamiate, coco-caprylate, coco-caprylate/caprate); alkanes (e.g., C9-C12 alkane, C13-15 alkane, coconut alkanes); squalene; dimethicone, and any mixtures thereof.

The deodorant composition can comprise a monoalkylglycol dialkyl acid ester solvent at concentrations ranging from about 0% to about 80%, preferably from about 0% to about 60%, more preferably from about 0% to about 50%, by weight of the composition. The modoalkylglycol dialkyl acid ester may be neopentyl glycol diheptanoate.

The deodorant composition can comprise a silicone solvent at concentrations ranging from about 0% to about 80%, preferably from about 0% to about 60%, more preferably from about 0% to about 50%, by weight of the composition. The volatile silicone of the solvent may be cyclic or linear.

Appropriate silicones may include volatile silicones, referring to those silicone materials which have measurable vapor pressure under ambient conditions. Nonlimiting examples of suitable volatile silicones are described in Todd et al., “Volatile Silicone Fluids for Cosmetics”, Cosmetics and Toiletries, 91:27-32 (1976), which descriptions are incorporated herein by reference. Preferred volatile silicone materials are those having from about 3 to about 7, preferably from about 4 to about 5, silicon atoms.

Cyclic volatile silicones are preferred for use in the antiperspirant compositions herein, and include those represented by the formula:

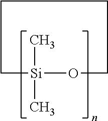

wherein n is from about 3 to about 7, preferably from about 4 to about 5, most preferably 5. These cyclic silicone materials will generally have viscosities of less than about 10 centistokes at 25° C.

Linear volatile silicone materials suitable for use in the antiperspirant compositions include those represented by the formula:

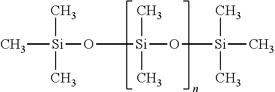

wherein n is from about 1 to about 7, preferably from about 2 to about 3. These linear silicone materials will generally have viscosities of less than about 5 centistokes at 25° C.

Specific examples of volatile silicone solvents suitable for use in the antiperspirant compositions include, but are not limited to, Cyclomethicone D-5 (commercially available from G. E. Silicones), Dow Corning 344, Dow Corning 345 and Dow Corning 200 (commercially available from Dow Corning Corp.), GE 7207 and 7158 (commercially available from General Electric Co.) and SWS-03314 (commercially available from SWS Silicones Corp.).

### Structurants

It may be desirable to make the deodorant composition in the form of a solid stick. The deodorant compositions may comprise a suitable concentration of structurants to help provide the compositions with the desired viscosity, rheology, texture and/or product hardness, or to otherwise help suspend any dispersed solids or liquids within the composition.

It is known that to formulate a solid antiperspirant or deodorant stick, the structurants generally have a melting point above 50° C. to provide a stable structure to the stick. The present inventors have discovered that a deodorant stick having at least about 25% of a liquid triglyceride, and that uses a primary structurant that has a melting point of at least about 50° C., alternatively from about 50° C. to 70° C., or alternatively from about 50° C. to about 75° C., while limiting the amount of secondary structurants having a melting point of at least about 60° C. to 8% or less, can result in a deodorant stick with a hardness from about 80 mm*10 to about 140 mm*10. Such a deodorant stick can include consumer-perceived natural ingredients, while offering a pleasant consumer experience in terms of its hardness.

The primary structurant may have a melting point of at least about 50° C., alternatively from about 50° C. to about 70° C., and alternatively from about 50° C. to about 75° C., and alternatively from about 60° C. to 80° C. A primary structurant is defined as the structurant that is present in the composition in the greatest amount (liquid triglycerides are not considered a structurant in this context). The composition may have just a single structurant, thus having only a primary structurant. The composition may have a primary structurant and then secondary structurants, those structurants that are used in a lesser amount than the primary structurant.

The primary structurant may comprise from about 5% to about 20%, alternatively about 7% to about 17% of the deodorant stick. The secondary structurants may cumulatively comprise about 12% or less, or about 8% or less of the deodorant stick, or less than about 5%, or less than about 3%, or less than about 1% of the deodorant stick. The deodorant stick may be free of or substantially free of any secondary structurants.

Some secondary structurants may have a melting point less than 60° C., and then remaining secondary structurants may have a melting point of at least about 60° C. The percentage of secondary structurants having a melting point less than 60° C. may not be as significant as the percentage of secondary structurants having a melting point of at least about 60° C., as the higher melting structurants are what contribute more to the hardness of the deodorant stick. The secondary structurants having a melting point of at least about 60° C. may cumulatively comprise 8% or less of the deodorant stick, less than about 5% of the deodorant stick, less than about 3% of the deodorant stick, or less than about 1% of the deodorant stick. The deodorant stick may be free of or substantially free of any secondary structurants having a melting point of at least about 60° C.

Waxes with melting points between 50° C. and 70° C. include Japan wax, lemon wax, grapefruit wax, beeswax, ceresine, paraffin, hydrogenated jojoba, ethylene glycol distearate, stearyl stearate, palmityl stearate, stearyl behenate, cetearyl behenate, hydrogenated high crucic acid rapeseed oil, and stearyl alcohol.

Waxes with melting points above 70° C. include ozokerite, candelilla, carnauba, espartograss, cork wax, guaruma, rice oil wax, sugar cane wax, ouricury, montan ester wax, sunflower wax, shellac, ozocerite, microcrystalline wax, sasol wax, polyethylenes, polymethylenes, ethylene glycol dipalmitate, ethylene glycol di(12-hydroxystearate), behenyl behenate, glyceryl tribehenate, hydrogenated castor oil (castor wax), and behenyl alcohol.

Waxes with melting points that could vary and possibly fall into either of the two previous groups (depending on factors such as chain length) include C18-C36 triglyceride, Fischer-Tropsch waxes, silicone waxes, C30-50 alkyl beeswax, C20-40 alkyl erucates, C18-38 alkyl hydroxy stearoyl stearates, C20-40 dialkyl esters of dimer acids, C16-40 alkyl stearates, C20-40 alkyl stearates, cetyl ester wax, and spermaceti.

Suitable gelling agents include fatty acid gellants such as fatty acid and hydroxyl or hydroxyl fatty acids, having from about 10 to about 40 carbon atoms, and ester and amides of such gelling agents. Non-limiting examples of such gelling agents include, but are not limited to, 12-hydroxystearic acid, 12-hydroxylauric acid, 16-hydroxyhexadecanoic acid, behenic acid, erucic acid, stearic acid, caprylic acid, lauric acid, isostearic acid, and combinations thereof. Preferred gelling agents are 12-hydroxystearic acid, esters of 12-hydroxystearic acid, amides of 12-hydroxystearic acid and combinations thereof.

These solid structurants include gelling agents, and polymeric or non-polymeric or inorganic thickening or viscosifying agents. Such materials will typically be solids under ambient conditions and include organic solids, crystalline or other gellants, inorganic particulates such as clays or silicas, or combinations thereof.

The concentration and type of solid structurant selected for use in the deodorant compositions will vary depending upon the desired product hardness, rheology, and/or other related product characteristics. For most structurants suitable for use herein, the total structurant concentration ranges from about 5% to about 35%, more typically from about 10% to about 30%, or from about 7% to about 20%, by weight of the composition.

Non-limiting examples of suitable structurants include stearyl alcohol and other fatty alcohols; hydrogenated castor wax (e.g., Castorwax MP80, Castor Wax, etc.); hydrocarbon waxes include paraffin wax, beeswax, carnauba, candelilla, spermaceti wax, ozokerite, ceresin, baysberry, synthetic waxes such as Fisher-Tropsch waxes, and microcrystalline wax; polyethylenes with molecular weight of 200 to 1000 daltons; solid triglycerides; behenyl alcohol, or combinations thereof. The deodorant stick may further comprise one or more structural elements selected from the group consisting of waxes, natural oils, coconut oil, fractionated coconut oil, jojoba seed oil, olive oil, soybean oil, sunflower oil, and combinations thereof.

Other non-limiting examples of primary structurants suitable for use herein are described in U.S. Pat. No. 5,976,514 (Guskey et al.) and U.S. Pat. No. 5,891,424 (Bretzler et al.), the descriptions of which are incorporated herein by reference.

Non-limiting examples of suitable additional structurants include stearyl alcohol and other fatty alcohols; hydrogenated castor wax (e.g., Castorwax MP80, Castor Wax, etc.); hydrocarbon waxes include paraffin wax, beeswax, carnauba, candelilla, spermaceti wax, ozokerite, ceresin, baysberry, synthetic waxes such as Fisher-Tropsch waxes, and microcrystalline wax; polyethylenes with molecular weight of 200 to 1000 daltons; and solid triglycerides; behenyl alcohol, or combinations thereof.

Other non-limiting examples of additional structurants suitable for use herein are described in U.S. Pat. No. 5,976,514 (Guskey et al.) and U.S. Pat. No. 5,891,424 (Bretzler et al.).

### Antimicrobials

The composition may include one or more antimicrobial compositions. For example, antimicrobials may include, without being limited to, octenidine dihydrochloride (octenidine HCl), hexamidine, polyvinyl formate, niacinamide, cinnamon essential oil, cinnamon bark essential oil, cinnamic aldehyde, piroctone olamine, polydialyldimethylammonium chloride, polyquaternium, and combinations thereof, baking soda, hexamidine, thymol, cinnamon essential oil, cinnamon bark essential oil, cinnamic aldehyde, polyvinyl formate, salicylic acid, niacinamide, phenoxyethanol, eugenol, linolenic acid, dimethyl succinate, citral, triethyl citrate, sepiwhite, a substituted or unsubstituted 2-pyridinol-N-oxide material (piroctone olamine), and combinations thereof. The deodorant stick may be free of or substantially free of a substituted or unsubstituted 2-pyridinol-N-oxide material.

In general, the total amount of antimicrobial used may be from about 0.03% to about 30%, by weight, of the deodorant. Some antimicrobials may be used in amounts as low as about 0.0.03%, by weight of the deodorant composition, such as if using octenidine dihydrochloride as the primary antimicrobial, while others could be as high as about 25% of the primary antimicrobial (primary antimicrobial being the antimicrobial present in the composition in the highest amount).

While numerous antimicrobials exhibit efficacy against two main bacteria strains that antiperspirants and deodorants try to address, due to regulatory and safety reasons, there are sometimes limits as to how much of a particular antimicrobial may be put into an antiperspirant or deodorant formula. Therefore, there may be a need for multiple antimicrobials to work together in a formula to deliver enough long-term odor protection.

The deodorant compositions as described herein can contain a structurant, an antimicrobial, a perfume, and additional chassis ingredient(s). The deodorant composition may further comprise other optional ingredient(s). The composition can be in the form of a gel. The composition can be in the form of a deodorant cream. The compositions can be in the form of a solid stick.

### Hardness

The deodorant compositions may have a product or stick hardness from about 60 mm*10 to about 160 mm*10, as measured by penetration with ASTM D-1321 needle (see Hardness test method below). The product hardness may be from about 80 to about 140 mm*10, and in others from about 85 to about 110 mm*10.

### Perfume

Perfumes are often a combination of many raw materials, known as perfume raw materials. Any perfume suitable for use in a deodorant composition may be used herein. The deodorant composition may be free of, or substantially free of a synthetic fragrance. A synthetic fragrance is one mostly derived through chemical synthesis where the starting material is no longer intact, but is converted to the new fragrance chemical.

A natural or essential oil fragrance is a result of natural sources wherein the fragrance material is not altered (chemically modified) but extracted from its natural source. These sources can include, but are not limited to, bark, flowers, blossoms, fruits, leaves, resins, roots, bulbs, and seeds. Natural or essential oils go through an extraction process instead of chemical synthesis. Extraction processes include, but are not limited to, maceration, solvent extraction, distillation, expression of a fruit peel, or effleurage.

### Additional Chassis Ingredients

### Starch

The deodorant composition may comprise a starch powder for dry feel or wetness absorption. Examples include but are not limited to arrowroot powder, tapioca starch, and corn starch.

### Non-Volatile Organic Fluids

Non-volatile organic fluids may be present, for example, in an amount of about 15% or less, by weight of the composition.

Non-limiting examples of nonvolatile organic fluids include mineral oil, PPG-14 butyl ether, isopropyl myristate, petrolatum, butyl stearate, cetyl octanoate, butyl myristate, myristyl myristate, C12-15 alkylbenzoate (e.g., Finsolv™), octyldodecanol, isostearyl isostearate, octododecyl benzoate, isostearyl lactate, isostearyl palmitate, and isobutyl stearate.

### Propellant

The compositions described herein can include a propellant. Some examples of propellants include compressed air, nitrogen, inert gases, carbon dioxide, and mixtures thereof. Propellants may also include gaseous hydrocarbons like propane, n-butane, isobutene, cyclopropane, and mixtures thereof. Halogenated hydrocarbons like 1,1-difluoroethane may also be used as propellants. Some non-limiting examples of propellants include 1,1,1,2,2-pentafluoroethane, 1,1,1,2-tetrafluoroethane, 1,1,1,2,3,3,3-heptafluoropropane, trans-1,3,3,3-tetrafluoroprop-1-ene, dimethyl ether, dichlorodifluoromethane (propellant 12), 1,1-dichloro-1,1,2,2-tetrafluoroethane (propellant 114), 1-chloro-1,1-difluoro-2,2-trifluoroethane (propellant 115), 1-chloro-1,1-difluoroethylene (propellant 142B), 1,1-difluoroethane (propellant 152A), monochlorodifluoromethane, and mixtures thereof. Some other propellants suitable for use include, but are not limited to, A-46 (a mixture of isobutane, butane and propane), A-31 (isobutane), A-17 (n-butane), A-108 (propane), AP70 (a mixture of propane, isobutane and n-butane), AP40 (a mixture of propane, isobutene and n-butane), AP30 (a mixture of propane, isobutane and n-butane), and 152A (1,1 difluoroethane). The propellant may have a concentration from about 15%, 25%, 30%, 32%, 34%, 35%, 36%, 38%, 40%, or 42% to about 70%, 65%, 60%, 54%, 52%, 50%, 48%, 46%, 44%, or 42%, or any combination thereof, by weight of the total fill of materials stored within the container.

### Other Optional Ingredients

The deodorant compositions may further comprise any optional material that is known for use in antiperspirant and deodorant compositions or other personal care products, or which is otherwise suitable for topical application to human skin.

One example of an optional ingredient is a scent expression material. Scent expression or release technology may be employed with some or all of the fragrance materials to define a desired scent expression prior to use and during use of the deodorant products. Such scent expression or release technology can include cyclodextrin complexing material, like beta cyclodextrin. Other materials, such as, for example, starch or silica-based matrices or microcapsules may be employed to “hold” fragrance materials prior to exposure to bodily-secretions (e.g., perspiration). The encapsulating material may have release mechanisms other than via a solvent; for example, the encapsulating material may be frangible, and as such, rupture or fracture with applied shear and/or normal forces encountered during application and while wearing. A microcapsule may be made from many materials, one example is polyacrylates.

Another example of optional materials are clay mineral powders such as talc, mica, sericite, silica, magnesium silicate, synthetic fluorphlogopite, calcium silicate, aluminum silicate, bentonite and montomorillonite; pearl pigments such as alumina, barium sulfate, calcium secondary phosphate, calcium carbonate, titanium oxide, finely divided titanium oxide, zirconium oxide, zinc oxide, hydroxy apatite, iron oxide, iron titrate, ultramarine blue, Prussian blue, chromium oxide, chromium hydroxide, cobalt oxide, cobalt titanate, titanium oxide coated mica; organic powders such as polyester, polyethylene, polystyrene, methyl methacrylate resin, cellulose, 12-nylon, 6-nylon, styrene-acrylic acid copolymers, poly propylene, vinyl chloride polymer, tetrafluoroethylene polymer, boron nitride, fish scale guanine, laked tar color dyes, laked natural color dyes; and combinations thereof.

Nonlimiting examples of other optional materials include emulsifiers, distributing agents, antimicrobials, pharmaceutical or other topical active, preservatives, surfactants, chelants, and so forth. Examples of such optional materials are described in U.S. Pat. No. 4,049,792 (Elsnau); U.S. Pat. No. 5,019,375 (Tanner et al.); and U.S. Pat. No. 5,429,816 (Hofrichter et al.); which descriptions are incorporated herein by reference.

The compositions may be free of and/or substantially free of aluminum. The compositions may be free of and/or substantially free of citric acid. The compositions may be free and/or substantially free of a starch or starch derivative. The compositions may be free of and/or substantially free of glycol. The composition can be substantially free of or free of parabens, dyes, and/or talc.

### Methods of Use

The deodorant compositions may be topically applied to the axilla or other area of the skin (e.g., chest, back, groin, feet, neck, inframammary fold) in any known or otherwise effective method for controlling malodor associated with perspiration. These methods comprise applying to the axilla or other area of the human skin an effective amount of the deodorant composition, typically about 3.5 to about 5 mg/cm², and more typically about 4 mg/cm². The deodorant composition is generally a leave-on composition that can provide lasting odor protection and freshness. The deodorant stick can be applied without white marks, in other words, it can go on clear and stay clear.

### EXAMPLES

The examples in Tables 3-6 were made and show inventive and comparative examples. The formulations are polar in nonpolar emulsions and were made in the following manner. The materials in the polar phase were mixed using conventional mixing techniques. The materials in the nonpolar phase were mixed with the materials in the emulsifier phase using conventional mixing techniques. To create the emulsion, the polar phase was added at a slow rate to the nonpolar phase with strong agitation of the nonpolar phase using overhead mixing. After all of the polar phase was added to the formulation, the emulsion was subsequently milled in a high shear homogenizer to create a solid gel polar in nonpolar emulsion.

All examples were placed at 49° C. for 1 week as an accelerated measure of stability to predict ambient stability. The Initial Complex Modulus and Complex Modulus after 1 week at 49° C. were measured with the Complex Modulus Rheology Method, described herein. Initial Complex Modulus is measured 24 hours after making or up to 14 days after making. A sample of the composition is placed in a Controlled Temperature, Controlled Humidity room set to 49° C. for 1 week. After 1 week at 49° C., the sample is pulled and equilibrated to room temperature (22° C. to 27° C.). Complex Modulus of the sample is measured after equilibrating to room temperature or up to 14 days after pulling the sample. The change in complex modulus is measured by the % Change in Complex Modulus.

$$\\ \% Change in Complex Modulus = \frac{\begin{matrix}Complex Modulus after 1 week at 49 ° C . - \\ Initial Complex Modulus\end{matrix}}{Initial Complex Modulus} \times 100$$

If the complex modulus of a product decreases by less than 15% over a 1-week period at 49° C., it is considered acceptable to consumers and likely indicates that the product will maintain consistent rheology and phase stability during shipping, handling, storage, and use throughout the product's shelf life.

The Surface Tension in Tables 3-6 were measured according to the Surface Tension Method, described herein. In Table 3, the polar phases were run for minimum 2 minutes with minimum 30 measured values and nonpolar phases were run for minimum 1 minutes with minimum 30 measured values. Surface tension values shown are the average of 3 samples. 2 wt % Acids in Water were run for minimum 1 minutes with minimum 30 measured values. The average of the last 5 values is used to calculate surface tension of a sample.

In Tables 3-6, the refractive index of each phase was measured according to the Refractive Index measurement. When refractive index is measured, most emulsifiers can be added to the nonpolar phase as the emulsifier will not change the refractive index of the nonpolar phase. If the emulsifier is added to the nonpolar phase for the refractive index measurement, it should be confirmed that the refractive index of the phase does not change. If the refractive index of the phase changes, then the emulsifier should not be included in the nonpolar phase for the measurement. For the measurements below, the emulsifier was determined to not change the refractive index of the nonpolar phase and therefore was added to the nonpolar phase for the refractive index measurement.

In Table 3, the difference in surface tension between the polar phase and nonpolar phase for Inventive Examples 1-4 was at least 20.0 mN/m and the surface tension of the primary carboxylic acid at a 2 wt % mixture in water is at least 69.8 mN/m. For Comparative Examples 5-6, the difference in surface tension between the polar phase and nonpolar phase was 18.6 mN/m and 19.6 mN/m, respectively, and the surface tension of the primary carboxylic acid at a 2 wt % mixture in water was 63.7 for both Comparative Examples 5-6.

As shown in Table 3, the Inventive Examples 1-4 had a decrease in the complex modulus of less than 15% over 1 week at 49° C., and are consumer acceptable. However, different properties of the carboxylic acid in Comparative Examples 5-6, caused the compositions to have a decrease in the complex modulus of more than 15% over 1 week at 49° C. (e.g., −39% and −37%, respectively), which is determined to be unacceptable to consumers. Thus, seemingly small differences in the surface tension of the primary carboxylic acid and/or the difference in surface tension between the polar phase and nonpolar phase can significantly impact the consistency of the rheology over time. It is anticipated that Examples 1-4 will have a consistent rheology over time and demonstrate long-term physical, thermal, and chemical stability, thus maintaining product performance over time. It is anticipated that these examples will be consumer acceptable.

TABLE 3

| | Inv. Ex. | Inv. Ex. | Inv. Ex. | Inv. Ex. | Comp. | Comp. |
|---|---|---|---|---|---|---|
| | 1 | 2 | 3 | 4 | Ex. 5 | Ex. 6 |
| | (wt. %) | (wt. %) | (wt. %) | (wt. %) | (wt. %) | (wt. %) |
| Polar Phase | | | | | | |
| Malic Acid ¹ | 4 | | 2.5 | | | |
| Glycolic Acid ² | | 4 | | | | |
| Succinic Acid ³ | | | | 3 | | |
| Mandelic Acid ⁴ | | | | | 2 | 2 |
| Sodium Citrate ⁵ | 1.95 | 2 | 1.6 | | 0.5 | 0.5 |
| Sodium Hydroxide ⁶ | | | | 0.2 | | |
| Propanediol (and) | 0.1 | 0.1 | 0.15 | 0.1 | 0.1 | 0.2 |
| Octenidine HCl ⁷ | | | | | | |
| PEG-8 (PEG 400) ⁸ | | | | | | 36 |
| PEG-12 (PEG 600) ⁹ | 25 | 25 | 27 | 25 | 25 | |
| Glycerin ¹⁰ | 15 | 15 | 9 | 15 | 15 | |
| Sodium Chloride ¹¹ | 0.5 | 0.5 | 0.5 | 0.5 | 0.5 | 0.5 |
| Water | Q.S. | Q.S. | Q.S. | Q.S. | Q.S. | Q.S. |
| Nonpolar Phase | | | | | | |
| Cyclopentasiloxane ¹² | 3.25 | 3.25 | 3.25 | 3.25 | 1.65 | 3.25 |
| Dimethicone ¹³ | 5 | 5 | 5 | 5 | 5 | 5 |
| Cyclopentasiloxane | 7 | 7 | 7 | 7 | 8.4 | 7 |
| (Emollient portion) ¹⁴ | | | | | | |
| Fragrance | 0.75 | 0.75 | 0.75 | 0.75 | 0.75 | 0.75 |
| Emulsifier Phase | | | | | | |
| PEG/PPG-18/18 | 1 | 1 | 1 | 1 | 1.2 | 1 |
| Dimethicone | | | | | | |
| (Emulsifier portion, | | | | | | |
| 12.5% dispersion) ¹⁴ | | | | | | |
| Polar Phase pH | 3.81 | 3.75 | 3.95 | 3.90 | 3.80 | 3.94 |
| Polar Phase | 1.411 | 1.411 | 1.401 | 1.406 | 1.406 | 1.401 |
| Refractive Index | | | | | | |
| Nonpolar Phase | 1.403 | 1.403 | 1.403 | 1.403 | 1.403 | 1.403 |
| Refractive Index | | | | | | |
| Polar Phase | 0.008 | 0.008 | −0.002 | 0.003 | 0.003 | −0.002 |
| Refractive Index - | | | | | | |
| Nonpolar Phase | | | | | | |
| Refractive Index | | | | | | |
| Initial Complex | 2306 | 2369 | 2106 | 2169 | 2718 | 1781 |
| Modulus (Pa) | | | | | | |
| Complex Modulus | 2373 | 2264 | 2130 | 2099 | 1670 | 1125 |
| after 1 week at 49° C. | | | | | | |
| (Pa) | | | | | | |
| % Change in | +2.9% | −4.4% | +1.1% | −3.2% | −39% | −37% |
| Complex Modulus | | | | | | |
| Polar Phase Surface | 41.6 | 39.4 | 38.9 | 41.6 | 37.5 | 38.5 |
| Tension (mN/m) | | | | | | |
| Non Polar Phase | 18.9 | 18.9 | 18.9 | 18.9 | 18.9 | 18.9 |
| Surface Tension | | | | | | |
| (mN/m) | | | | | | |
| Polar Phase Surface | 22.7 | 20.5 | 20.0 | 22.7 | 18.6 | 19.6 |
| Tension - Nonpolar | | | | | | |
| Phase Surface | | | | | | |
| Tension (mN/m) | | | | | | |
| Primary Carboxylic | Malic | Glycolic | Malic | Succinic | Mandelic | Mandelic |
| Acid | Acid | Acid | Acid | Acid | Acid | Acid |
| Surface Tension of | 71.8 | 71.1 | 71.8 | 69.8 | 63.7 | 63.7 |
| Primary Carboxylic | | | | | | |
| Acid when measured | | | | | | |
| at 2 wt % in Water | | | | | | |
| (mN/m) | | | | | | |
| Primary Carboxylic | Yes | Yes | Yes | Yes | No | No |
| Acid has a CLogD | | | | | | |
| less than −0.5 at a pH | | | | | | |
| from 3.0 to 5.0 | | | | | | |
| Primary Carboxylic | Yes | Yes | Yes | Yes | No | No |
| Acid has a Log S | | | | | | |
| greater than 2.5 at a | | | | | | |
| pH from 3.0 to 5.0 | | | | | | |
| at 25° C. | | | | | | |

Inventive Examples 7-10, in Table 4, the surface tension of the primary carboxylic acid at a 2 wt % mixture in water is at least 69.8 mN/m. For Comparative Example 11, the surface tension of the primary carboxylic acid at a 2 wt % mixture in water was 63.7.

In Table 4, Inventive Examples 7-10 had a decrease in the complex modulus of less than 15% over 1 week at 49° C., which was determined to be acceptable to consumers. Comparative Example 11 had a decrease in the complex modulus of 31%, which is more than 15% over 1 week at 49° C., which was determined to be unacceptable to consumers.

It is anticipated that Examples 7-10 will have good product performance as made. These examples are also expected to demonstrate long-term physical, thermal, and chemical stability, thus maintaining product performance over time. It is anticipated that these examples and will be consumer acceptable.

TABLE 4

| | Inventive | Inventive | Inventive | Inventive | Comp. |
|---|---|---|---|---|---|
| | Ex. 7 | Ex. 8 | Ex. 9 | Ex. 10 | Ex. 11 |
| | (wt. %) | (wt. %) | (wt. %) | (wt. %) | (wt. %) |
| Polar Phase | | | | | |
| Malic Acid ¹ | 7.5 | 6 | | | |
| Glycolic Acid ² | | | 5.5 | | |
| Mandelic Acid ⁴ | | | | | 4 |
| Citric Acid ¹⁵ | | | | 5.5 | |
| Sodium Citrate ⁵ | 4.8 | 3.8 | 4.1 | 3.6 | 1.4 |
| Propanediol (and) | 0.15 | 0.15 | 0.15 | 0.15 | 0.1 |
| Octenidine HCl ⁷ | | | | | |
| PEG-12⁹ | 26 | 26 | 26 | 26 | 26 |
| Propylene Glycol ¹⁶ | 10 | 10 | 10 | 10 | 10 |
| Sodium Chloride ¹¹ | 0.5 | 0.5 | 0.5 | 0.5 | 1 |
| Water | Q.S. | Q.S. | Q.S. | Q.S. | Q.S. |
| Nonpolar Phase | | | | | |
| Cyclopentasiloxane ¹² | 3.25 | 3.25 | 3.25 | 3.25 | 3.25 |
| Dimethicone ¹³ | 5 | 5 | 5 | 5 | 5 |
| Cyclopentasiloxane | 7 | 7 | 7 | 7 | 7 |
| (Emollient portion) ¹⁴ | | | | | |
| Fragrance | 0.75 | 0.75 | 0.75 | 0.75 | 0.75 |
| Emulsifier Phase | | | | | |
| PEG/PPG-18/18 | 1 | 1 | 1 | 1 | 1 |
| Dimethicone | | | | | |
| (Emulsifier portion, | | | | | |
| 12.5% dispersion) ¹⁴ | | | | | |
| Polar Phase pH | 3.98 | 3.97 | 4.02 | 4.02 | 4.02 |
| Polar Phase | 1.413 | 1.409 | 1.407 | 1.409 | 1.405 |
| Refractive Index | | | | | |
| Nonpolar Phase | 1.403 | 1.403 | 1.403 | 1.403 | 1.403 |
| Refractive Index | | | | | |
| Polar Phase | 0.011 | 0.006 | 0.004 | 0.006 | 0.002 |
| Refractive Index − | | | | | |
| Nonpolar Phase | | | | | |
| Refractive Index | | | | | |
| Initial Complex | 1993 | 2179 | 2027 | 1963 | 1730 |
| Modulus (Pa) | | | | | |
| Complex | 2027 | 1914 | 2108 | 1862 | 1192 |
| Modulus after 1 | | | | | |
| week at 49° C. (Pa) | | | | | |
| % Change in | +1.7% | −12% | +4.0% | −5.1% | −31% |
| Complex Modulus | | | | | |
| Primary Carboxylic | Malic | Malic | Glycolic | Citric | Mandelic |
| Acid | Acid | Acid | Acid | Acid | Acid |
| Surface Tension of | 71.8 | 71.8 | 71.1 | 71.3 | 63.7 |
| Primary Carboxylic | | | | | |
| Acid when | | | | | |
| measured at 2 wt % | | | | | |
| in Water (mN/m) | | | | | |
| Primary Carboxylic | Yes | Yes | Yes | Yes | No |
| Acid has a CLogD | | | | | |
| less than −0.5 at a | | | | | |
| pH from 3.0 to 5.0 | | | | | |
| Primary Carboxylic | Yes | Yes | Yes | Yes | No |
| Acid has a Log S | | | | | |
| greater than | | | | | |
| 2.5 at a pH from | | | | | |
| 3.0 to 5.0 at 25° C. | | | | | |

In Table 5, Inventive Examples 12-13 had a decrease in the complex modulus that was less than 15% over 1 week at 49° C., which was determined to be acceptable to consumers. It is anticipated that Examples 12-13 will have good product performance as made. These examples are also expected to demonstrate long-term physical, thermal, and chemical stability, thus maintaining product performance over time. It is anticipated that these examples and will be consumer acceptable.

TABLE 5

| | Inventive | Inventive |
|---|---|---|
| | Ex. 12 | Ex. 13 |
| | (wt. %) | (wt. %) |
| Polar Phase | | |
| Malic Acid ¹ | 6 | |
| Glycolic Acid ² | | 5.5 |
| Sodium Citrate ⁵ | 3.7 | 4 |
| Propanediol (and) | 0.15 | 0.15 |
| Octenidine HCl ⁷ | | |
| PEG-12 ⁹ | 26 | 26 |
| 1,3-Propanediol ¹⁷ | 10 | 10 |
| Sodium Chloride ¹¹ | 0.5 | 0.5 |
| Water | Q.S. | Q.S. |
| Nonpolar Phase | | |
| Cyclopentasiloxane ¹² | 3.25 | 3.25 |
| Dimethicone ¹³ | 5 | 5 |
| Cyclopentasiloxane | 7 | 7 |
| (Emollient portion) ¹⁴ | | |
| Fragrance | 0.75 | 0.75 |
| Emulsifier Phase | | |
| PEG/PPG-18/18 Dimethicone | 1 | 1 |
| (Emulsifier portion, 12.5% | | |
| dispersion) ¹⁴ | | |
| Polar Phase pH | 4.01 | 4.04 |
| Polar Phase Refractive Index | 1.409 | 1.406 |
| Nonpolar Phase Refractive Index | 1.403 | 1.403 |
| Polar Phase Refractive Index − | 0.006 | 0.003 |
| Nonpolar Phase Refractive Index | | |
| Initial Complex Modulus (Pa) | 1666 | 1697 |
| Complex Modulus after 1 week | 1502 | 1557 |
| at 49° C. (Pa) | | |
| % Change in Complex Modulus | −9.8% | −9.0% |
| Primary Carboxylic Acid | Malic | Glycolic |
| | Acid | Acid |
| Surface Tension of Primary | 71.8 | 71.1 |
| Carboxylic Acid when measured | | |
| at 2 wt % in Water (mN/m) | | |
| Primary Carboxylic Acid has a | Yes | Yes |
| CLogD less than −0.5 at a pH | | |
| from 3.0 to 5.0 | | |
| Primary Carboxylic Acid has a | | |
| Log S greater than 2.5 at a pH | Yes | Yes |
| from 3.0 to 5.0 at 25° C. | | |

In Table 6. Comparative Examples 14-16 had a decrease in the complex modulus of more than 15% over 1 week at 49° C. which is determined to be unacceptable to consumers and therefore, these examples are not preferred.

TABLE 6

| | Comp. | Comp. | Comp. |
|---|---|---|---|
| | Ex. 14 | Ex. 15 | Ex. 16 |
| | (wt. %) | (wt. %) | (wt. %) |
| Polar Phase | | | |
| Mandelic Acid ⁴ | 4 | 2 | 2 |
| Sodium Citrate ⁵ | 1 | 0.5 | 0.5 |
| Propanediol (and) | 0.1 | 0.1 | 0.1 |
| Octenidine HCl ⁷ | | | |
| PEG-8 ⁸ | | 36 | |
| PEG-12 ⁹ | 30 | | 25 |
| Glycerin ¹⁰ | 10 | | 15 |
| Sodium Chloride ¹¹ | 0.5 | 0.5 | 0.5 |
| Water | Q.S. | Q.S. | Q.S. |
| Nonpolar Phase | | | |
| Cyclopentasiloxane ¹² | 3.25 | 3.25 | 6.65 |
| Dimethicone ¹³ | 5 | 5 | |
| Cyclopentasiloxane | 7 | 7 | 8.4 |
| (Emollient portion) ¹⁴ | | | |
| Fragrance | 0.75 | 0.75 | 0.75 |
| Emulsifier Phase | | | |
| PEG/PPG-18/18 Dimethicone | 1 | 1 | 1.2 |
| (Emulsifier portion, 12.5% | | | |
| dispersion) ¹⁴ | | | |
| Polar Phase pH | 3.90 | 3.89 | 3.76 |
| Initial Complex Modulus (Pa) | 2135 | 2382 | 2206 |
| Complex Modulus | 1452 | 1460 | 1327 |
| after 1 week at 49° C. (Pa) | | | |
| % Change in Complex Modulus | −32% | −39% | −40% |
| Primary Carboxylic Acid | Mandelic | Mandelic | Mandelic |
| | Acid | Acid | Acid |
| Surface Tension of Primary | 63.7 | 63.7 | 63.7 |
| Carboxylic Acid when measured | | | |
| at 2 wt % in Water (mN/m) | | | |
| Primary Carboxylic Acid has a | No | No | No |
| CLogD less than −0.5 at a pH | | | |
| from 3.0 to 5.0 | | | |
| Primary Carboxylic Acid has a | No | No | No |
| Log S greater than 2.5 at a pH | | | |
| from 3.0 to 5.0 at 25° C. | | | |

### Suppliers for Examples in Table 3 to Table 6

1. Malic Acid Fine Granular available from Primary Products Ingredients Americas LLC
2. Glypure® 70 Glycolic Acid available from Chemours® (70% solution of Glycolic Acid in Water
3. Succinic Acid available from VWR®
4. TEGO® RenewHA Mandelic available from Evonik®
5. Trisodium Citrate Dihydrate available from Jungbunzlauer®
6. Liquid Caustic Soda 50% Membrane available from Westlake® (50% solution of Sodium Hydroxide in Water)
7. Sensidin™ Pure available from Ashland®
8. Carbowax™ Sentry™ Polyethylene Glycol 400 NF available from Dow®
9. Carbowax™ Sentry™ Polyethylene Glycol 600 NF available from Dow®
10. GL99.7 USP available from Cremer
11. Sodium Chloride available from Morton® Salt
12. SF1202 available from Momentive®
13. Xiameter® PMX-200 Silicone Fluid 10 cst available from Dow®
14. Dowsil™ 5225C Formulation Aid available from Dow (12.5% dispersion of PEG/PPG-18/18 Dimethicone in Cyclopentasiloxane)
15. Citric Acid USP Anhydrous Fine Granular available from Archer Daniels Midland Company
16. PuraGuard™ Propylene Glycol USP available from Dow®
17. Zemea® Propanediol available from Covation®

### MIC of Various Carboxylic Acids

The following MIC (Minimum Inhibitory Concentration) was done on various carboxylic acids. The MIC is the lowest concentration of active compound at which no growth of the microorganism is observed macroscopically. The MIC of the carboxylic acids below was tested at pH 5 against a mock community of five underarm species (3 Staphylococcus and 2 Corynebacterium). Tryptic Soy Broth is used in the measurements below as a standard diluent for testing MIC.

Initial concentration is the highest concentration tested in this experiment. The MIC is determined by optical density at a wavelength of 600 nm using a Molecular Devices SpectraMax iD5. The Optical Density MIC 95 is defined as the concentration of acid that demonstrates less than 5% of the total optical density achieved in the absence of acid and is used to measure the MIC. Several acids demonstrated Optical Density MIC 95 at 1000 ppm concentration in Tryptic Soy Broth. Other acids demonstrated Optical Density MIC 95 at even lower concentrations in Tryptic Soy Broth. The results shown in Table 7 below indicate that carboxylic acids have the ability to inhibit bacteria growth. The MIC is defined as the lowest concentration of compound that inhibits the growth of the bacterial population of interest to greater than or equal to 95% relative to the no treatment control (i.e. media only controls) after ˜24 hours of incubation at 37° C.

TABLE 7

| | | | Optical |
|---|---|---|---|
| | | Initial | Density |
| | | Concentration, | MIC 95, |
| | Diluent | pH 5 | pH 5 |
| Fumaric Acid | Tryptic Soy Broth | 1000 ppm | 250 ppm |
| Lactic Acid | Tryptic Soy Broth | 1000 ppm | 1000 ppm |
| Malic Acid | Tryptic Soy Broth | 1000 ppm | 500 ppm |
| Maleic Acid | Tryptic Soy Broth | 1000 ppm | 1000 ppm |
| Mandelic Acid | Tryptic Soy Broth | 1000 ppm | 1000 ppm |
| Salicylic Acid | Tryptic Soy Broth | 1000 ppm | 125 ppm |
| Succinic Acid | Tryptic Soy Broth | 1000 ppm | 1000 ppm |
| Tartaric Acid | Tryptic Soy Broth | 1000 ppm | 250 ppm |

### Test Methods

### Complex Modulus Rheology Method

Dynamic oscillatory testing is used to measure the complex modulus (G*) of a high internal phase polar in nonpolar emulsion. A parallel plate geometry is used that includes serrated top and bottom plates. Samples are prepared by placing the sample on the serrated plate geometry with minimal shear and performing a stress sweep measurement. The stress is applied to the sample starting from a low stress and increased until the sample yields or flows. While the stress sweep is running, the complex modulus is measured by the rheometer. Complex modulus (G*) is the relationship between the elastic and viscous modulus and indicates the overall structure of a gel. The Complex Modulus (G*) is outputted by standard rheology programs utilized on rheometers at each value of stress tested. The measurement is run at a controlled temperature of 22° C. using a controlled temperature geometry and a water bath to maintain the controlled temperature. A 35 mm serrated plate is used. Gap is set at 0.5 mm. After the samples is loaded, the sample is trimmed per typical trimming procedures used in rheology to ensure the sample edge is flush with the geometry. The oscillatory stress sweep is run from 1 Pa to 800 Pa. The complex modulus reported is the plateau complex modulus. The plateau complex modulus reported is the complex modulus at which the slope of the complex modulus vs. stress is 0.

### Surface Tension Method

Surface Tension is measured by ASTM D1331-20 Method C-Surface Tension by Willhelmy plate. For accurate measurement, the Surface Tension measurement should be allowed to equilibrate over the course of at least 1 minute, or at least 2 minutes, or longer depending on the sample. Properly equilibrated surface tension measurement is evaluated by not seeing a change in the surface tension measurement across multiple data points at the end of the measurement time period. The sample is run for a longer period of time if the surface tension measurement is continuing to change across multiple data points at the end of the measurement time period. A minimum of 30 measured values are collected during the measurement time period. The average of the last 5 values collected is used to calculate surface tension of a sample. Surface tension values shown are the average of 3 samples.

### pH Method

First, calibrate the Mettler Toledo Seven Compact pH meter. Do this by turning on the pH meter and waiting for 30 seconds. Then take the electrode out of the storage solution, rinse the electrode with distilled water, and carefully wipe the electrode with a scientific cleaning wipe, such as a Kimwipe®. Submerse the electrode in the pH 4 buffer and press the calibrate button. Wait until the pH icon stops flashing and press the calibrate button a second time. Rinse the electrode with distilled water and carefully wipe the electrode with a scientific cleaning wipe. Then submerse the electrode into the pH 7 buffer and press the calibrate button a second time. Wait until the pH icon stops flashing and press the calibrate button a third time. Rinse the electrode with distilled water and carefully wipe the electrode with a scientific cleaning wipe. Then submerse the electrode into the pH 10 buffer and press the calibrate button a third time. Wait until the pH icon stops flashing and press the measure button. Rinse the electrode with distilled water and carefully wipe with a scientific cleaning wipe. Submerse the electrode into the testing sample and press the read button. Wait until the pH icon stops flashing and record the value. If the deodorant composition is a solid, measure the pH of the polar phase prior to adding this phase to the nonpolar phase.

### Interfacial Tension

Interfacial Tension is measured by ISO-19403-4-2017 Pendant Drop method.

### Refractive Index—Measurement of Refractive Index (RI)

A refractometer is the instrument used to measure refractive index (RI). A refractometer measures the extent to which light is bent when it moves from air into a sample and is typically used to determine the refractive index of a liquid sample. Refractive index can be measured by any method suitable to provide an accurate refractive index, with a minimum resolution of 0.0001 nD.

There are four main types of refractometers, traditional handheld refractometers, digital handheld refractometers, laboratory or Abbe refractometer and inline process refractometers. A digital handheld refractometer (e.g. AR200 Digital Handheld Refractometer, Reichert) can be used, whereas the instrument is calibrated with a standard solution (typically water). A few drops of the sample are added to the sample plate and the measurement is taken. The refractive index for the sample is recorded.

### Clarity Assessment—Measurement of % Transmittance (% T)

Clarity can be measured by % Transmittance (% T) using Ultra-Violet/Visible (UV/VI) spectrophotometry which determines the transmission of UV/VIS light through a sample. A light wavelength of 600 nm has been shown to be adequate for characterizing the degree of light transmittance through a sample. Typically, it is best to follow the specific instructions relating to the specific spectrophotometer being used. In general, the procedure for measuring percent transmittance starts by setting the spectrophotometer to 600 nm. Then a calibration “blank” is run to calibrate the readout to 100 percent transmittance. A single test sample is then placed in a cuvette designed to fit the specific spectrophotometer and care is taken to ensure no air bubbles are within the sample before the % T is measured by the spectrophotometer at 600 nm. An example of equipment used for measurement is X-Rite Ci7800 Bench Top Spectrophotometer. The compositions may have a percent transmittance (% T) of at least about 80% transmittance at 600 nm.

### Minimum Inhibitory Concentration (MIC)

The MIC or minimum inhibitory concentration test determines antimicrobial activity of a material against a specific bacteria.

The most commonly employed methods are the tube dilution method and agar dilution method. Test products that are not clear or precipitate the growth media are tested by agar dilution method which is similar to the tube dilution method except dilutions are plated on agar.

The tube dilution test is the standard method for determining levels of microbial resistance to an antimicrobial agent. Serial dilutions of the test agent are made in a liquid microbial growth medium which is inoculated with a standardized number of organisms and incubated for a prescribed time. The lowest concentration (highest dilution) of test agent preventing appearance of turbidity (growth) is considered to be the minimal/minimum inhibitory concentration (MIC). At this dilution the test agent is bacteriostatic. Turbidity is measured with an optical reader (for example a Molecular Devices SpectraMax iD5) at 600 nm. This measurement is referred to as optical density.

### Finished Product Soleris Method

Sample preparation is done in the following manner. The product to be tested is weighed into a vial at a ratio of 1 part product to 10 parts Artificial Eccrine, custom pH 6, non-stabilized as obtained by Pickering Laboratories (cat. No. 1700-0023). In some cases where products have a higher hostility, the dilution in a particular study may be 1 part deodorant product to 100 parts Artificial Eccrine, custom pH 6, non-stabilized. Products are heated to 72° C. for 30 minutes to melt solid products and facilitate dispersion into the artificial eccrine. Vial is agitated vigorously to disperse the solids, using a Vortex Genie Agitator or comparable equipment. There may be some insoluble media present after dispersion. The dilutions remainder of the procedure is described below in “Analyzing via Soleris”.

### SOLERIS Swab Collection (3 Total Per Underarm):

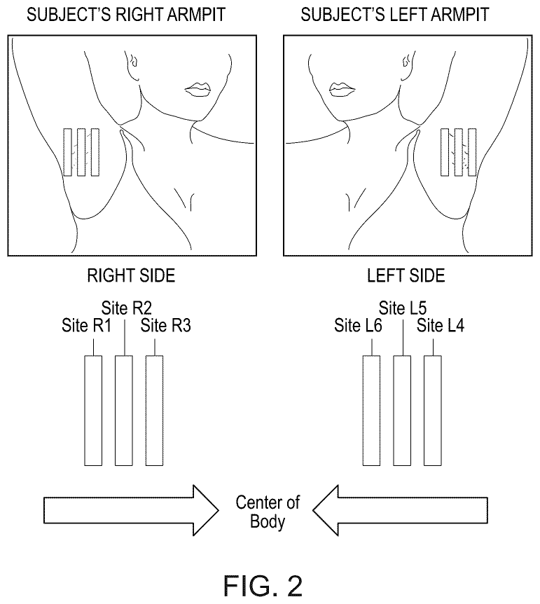

The sampling method is described as follows and references FIG. 2:
1. The subject's armpit will need to be accessible.
2. Practice holding the swab to identify a way that will be comfortable for you. DO NOT touch the swab tip after removal from properly labeled sterile transport tube.
3. Please note: for swabbing you will work from the outside towards the inside body for each armpit. For each subject, you will swab the RIGHT armpit, then the LEFT armpit, according to FIG. 2.
4. The technician will put on a new pair of gloves.
5. Samples will be collected from the RIGHT armpit.
6. The Copan sterile swab is dipped in distilled water.
7. Identify the midline of the Subject's RIGHT armpit. Starting just left of the midline (Site R1) of the Subject's RIGHT armpit, press the swab firmly against the skin and in a vertical direction run the swab up and down for 10 strokes traveling approximately 4 inches per stroke. One stroke is 1 upward motion and 1 downward motion. After each stroke, rotate the swab approximately one-half turn.
8. Once swabbing in Site R1 is complete, the swab will be returned to the labeled sterile transport tube and placed immediately on wet ice.
9. A second Copan swab will be removed from labeled transport tube and dipped in distilled water.
10. Identify the midline (Site R2) of the Subject's RIGHT armpit. Press the swab firmly against the skin and in a vertical direction run the swab up and down the skin for 10 strokes. 1 stroke is (one upward motion and 1 downward motion). After each stroke rotate the swab approximately one-half turn.
11. Once swabbing in Site R2 is complete, the swab will be returned to the labeled sterile transport tube and placed immediately on wet ice.
12. A third Copan swab will be removed from labeled transport tube and dipped in distilled water.
13. Identify the midline of the Subject's RIGHT armpit. Starting just right of the midline (Site R3) of the Subject's RIGHT armpit, press the swab firmly against the skin and in a vertical direction run the swab up and down the skin for 10 strokes. 1 stroke is (one upward motion and 1 downward motion). After each stroke rotate the swab approximately one-half turn.
14. Once swabbing in Site R3 is complete, the swab will be returned to the labeled sterile transport tube and placed immediately on wet ice.
15. Samples will be collected from the LEFT armpit.
16. The Copan sterile swab is dipped in distilled water.
17. Identify the midline of the Subject's LEFT armpit. Starting just right of the midline (Site LA) of the Subject's RIGHT armpit, press the swab firmly against the skin and in a vertical direction run the swab up and down for 10 strokes. One stroke is 1 upward motion and 1 downward motion. After each stroke, rotate the swab approximately one-half turn.
18. Once swabbing in Site LA is complete, the swab will be placed in the designated swab tube and the tube will be placed immediately on wet ice.
19. A second Copan swab will be dipped in distilled water.
20. Identify the midline (Site L5) of the Subject's LEFT armpit. Press the swab firmly against the skin and in a vertical direction run the swab up and down the skin for 10 strokes. 1 stroke is (one upward motion and 1 downward motion). After each stroke rotate the swab approximately one-half turn.
21. Once swabbing in Site L5 is complete, the swab will be placed in the swab tube and the tube will be placed immediately on wet ice.
22. A third Copan swab will be dipped in distilled water.
23. Identify the midline of the Subject's LEFT armpit. Starting just left of the midline (Site L6) of the Subject's RIGHT armpit, press the swab firmly against the skin and in a vertical direction run the swab up and down the skin for 10 strokes. 1 stroke is (one upward motion and 1 downward motion). After each stroke rotate the swab approximately one-half turn.
24. Once swabbing in Site L6 is complete, the swab will be placed in the swab tube and the tube will be placed immediately on wet ice.
25. Once the subject swabbing is complete, all swabs in their tubes will be placed in a Ziploc bag labeled with the Subject Number and SOLERIS and refrigerated.
26. The technician will change gloves in between subjects.

### Analyzing Via Soleris

S. epidermidis grown overnight on tryptic soy agar (37° C.)
Colony from agar is suspended in 25 ml tryptic soy broth in baffled flask, and incubated overnight on shaking platform (37° C.)
1 ml of overnight culture is diluted into 9 ml of sterile H₂O (this serves as the inoculum for experimental formulas)
For testing liquid products, 5 ml of experimental formula transferred to 15 ml conical tube. For testing solid formulations, a 48 or 96-well tissue culture plates is used, with each well containing 100 μl of solidified formulations. Alternately, for solid formulations, collect 10-20g of product and apply 10-20 g of water and let sit at least 1 hour. Moderate agitation may be used to release actives from sample.
50 μl of inoculum (˜10⁶ bacterial cfus) added to 5 ml of formula in conical tube (or 1-5 μl of inoculum in culture plates), and the liquid mixture is incubated on rocking platform or stationary in case of culture plates at room temperature
At 0, 3, 6 and 24 h, 100 μl of mixture is transferred to separate Soleris NF-TVC vial, mixed by inversion and placed into Soleris hardware for continuous 48 h analysis at 34° C. per vendors instructions. Alternatively, the mixture can be subjected traditional serial dilution, plating and incubation to determine surviving cfu/ml.
Soleris analysis provides Detection Time (DT) which is inversely proportional to living microbial biomass in mixture
Extension of DT vs base formula control reflects relative microbial hostility of experimental formula and reduction of living microbial biomass

### Hardness Test Method—Penetration Measurement for Deodorant Finished Products

The penetration test is a physical test method that provides a measure of the firmness of waxy solids and extremely thick creams and pastes with penetration values not greater than 250 when using a needle for D1321. The method is based on the American Society for Testing and Materials Methods D-5, D1321 and D217 and DIN 51 579 and is suitable for all solid antiperspirant and deodorant products.

A needle or polished cone of precisely specified dimensions and weight is mounted on the bottom of a vertical rod in the test apparatus. The sample is prepared as specified in the method and positioned under the rod. The apparatus is adjusted so that the point of the needle or cone is just touching the top surface of the sample. Consistent positioning of the rod is critical to the measured penetration value. The rod is then released and allowed to travel downward, driven only by the weight of the needle (or cone) and the rod. Penetration is the tenths of a millimeter travelled following release.

### Apparatus Suggested Type (or Equivalent)

Penetrometer with Timer

Penetrometer Suitable For ASTM D-5 and D-1321 methods; Examples: Precision or Humboldt Universal Penetrometer (Humboldt Manufacturing, Schiller Park, IL USA) or Penetrometer Model PNR10 or PNR 12 (Petrolab USA or PetroTest GmbH).

### Penetration Needles ANTIPERSPIRANT or DEODORANT SOLIDS can use:

Needles as specified for ASTM Method D-5, NIST Certified, Fisher Scientific #01-512.
Needles as specified for ASTM Method D 1321/DIN 51 579, Officially certified, Taper-Tipped needle, No. H-1310, Humboldt Mfg.
   General Instructions—All Penetrometers-Keep the instrument and needles/probes clean at all times, free from dust and grime. When not in use, store needles in a suitable container to avoid damage. Periodic calibration should confirm:
   Electronic Timer is correctly set. Verify against an independent stopwatch if unsure.
   Shaft falls without visible signs of frictional resistance.
   Ensure the total weight of the shaft and needle is 50±0.2 grams when the shaft is in free fall. Note: for modern, automated or digital systems this may be performed automatically and confirmed through annual calibration.
   At time of use confirm:
   Electronic Timer is correctly set to 5.0 seconds.
   The appropriate needle is installed and is clean, straight and without obvious defects (visual inspection)
   The penetrometer is level and the shaft is clean, straight and falls freely (visual inspection) Once level, avoid shifting the position of the unit to maintain level.

### Sample Preparation and Measurement

1. On a deodorant stick that has cooled ambiently to a temperature between 22° C. and 26° C. for at least 24 hours, slice off top ½ inch of product to achieve a flat surface with a wire cutter drawn across the upper lip of the canister.
2. For the first sample to be tested, lubricate the needle by gently wiping with a lint-free tissue coated with a small amount of the product to be tested. This small amount is typically taken from the shaved top.
3. Place the canister in the appropriate location for the measurement. Locate the sample so the needle will penetrate the product 9-11 mm from the inside of the canister wall on the long axis.
4. Using the coarse and fine adjustments, align the height of the penetrometer mechanism head so that the point of the penetrating needle is just touching the surface of the sample. A weak light at the side of the penetrometer which casts a shadow of the needle on the surface of the sample may be helpful in determining this contact. When a light area on the sample cannot be seen at the end of the tip of the needle's shadow, the needle height over the sample is correctly adjusted. The light should not be strong enough to heat or melt the sample surface. The needle should be just close enough to scratch the sample surface.
5. Perform the penetration measurement at this location by releasing the needle. Record the result.
6. Repeat Steps 2 through 4 at the other test point, i.e., at the other point 9-11 mm inside of the canister wall on the long axis.
   To report results, units for penetration are tenths of a millimeter ( 1/10 mm=100 microns). For example, a result of 80 units is 80 mm*10 or 8 mm. Report the average results of at least 4 total measurements from 2 different sticks, report to the nearest tenth of a millimeter.

### Combinations

A. A deodorant composition, said composition comprising:
   a primary carboxylic acid, wherein the primary carboxylic acid has a C Log D less than −0.5 at a pH from 3.0 to 5.0;
   wherein the deodorant composition is a polar in nonpolar emulsion, comprising a polar phase, a nonpolar phase and an emulsifier phase;
   wherein the polar phase has a refractive index that is at most 0.01 different than the nonpolar phase;
   wherein the composition is free of aluminum;
   wherein the polar phase has a surface tension that is at least 20.0 mN/m greater than the nonpolar phase.
B. The deodorant composition according to paragraph A, wherein the primary carboxylic acid has a surface tension that is at least 69.8 mN/m when measured in a 2 wt % mixture in water.
C. The deodorant composition according to paragraphs A and B, wherein the primary carboxylic acid has a Log S greater than 2.5 mol/L at a pH from 3.0 to 5.0 at 25° C.
D. The deodorant composition according to paragraphs A to C, wherein the primary carboxylic acid has a C Log D less than −0.5 at a pH from 3.0 to 4.5;
E. The deodorant composition according to paragraphs A to D, wherein the primary carboxylic acid has a Log S greater than 2.5 mol/L at a pH from 3.0 to 4.5 at 25° C.
F. The deodorant composition according to paragraphs A to E, wherein the primary carboxylic acid is a deodorant active.
G. The deodorant composition according to paragraphs A to F, wherein the primary carboxylic acid is selected from malic acid, glycolic acid, citric acid, succinic acid, and combinations thereof.
H. The deodorant composition according to paragraphs A to G, wherein the polar phase has a refractive index that is at most 0.008 different than the nonpolar phase.
I. The deodorant composition according to paragraphs A to H, wherein the polar phase has a refractive index that is at most 0.004 different than the nonpolar phase.
J. The deodorant composition according to paragraphs A to I, wherein the polar in nonpolar emulsion is a polar in silicone emulsion.
K. The deodorant composition according to paragraphs A to J, wherein the polar in nonpolar emulsion is a water in oil emulsion.
L. The deodorant composition according to paragraphs A to K, wherein the polar in nonpolar emulsion is a water in silicone emulsion.
M. The deodorant composition according to paragraphs A to L, wherein the polar phase comprises at least about 75% of the composition.
N. The deodorant composition according to paragraphs A to M, wherein the polar phase comprises at least about 82% of the composition.
O. The deodorant composition according to paragraphs A to N, wherein the primary carboxylic acid has a C Log D less than −0.6 at a pH from 3.0 to 5.0.
P. The deodorant composition according to paragraphs A to O, wherein the primary carboxylic acid has a C Log D less than −0.8 at a pH from 3.0 to 5.0.
Q. The deodorant composition according to paragraphs A to P, wherein the polar phase has a surface tension that is at least 21 mN/m greater than the surface tension of the nonpolar phase.
R. The deodorant composition according to paragraphs A to Q, wherein the polar phase has a surface tension that is at least 22.7 mN/m greater than the surface tension of the nonpolar phase.
S. The deodorant composition according to paragraphs A to R, wherein the polar phase has a refractive index that is at most 0.005 different than the polar phase.
T. The deodorant composition according to paragraphs A to S, wherein the composition has a percent transmittance (% T) of at least about 80% transmittance at 600 nm.
U. The deodorant composition according to paragraphs A to T, wherein the composition comprises glycerin.
V. The deodorant composition according to paragraphs A to U, wherein the composition comprise PEG.
W. The deodorant composition according to paragraphs A to V, further comprising an antimicrobial selected from the group consisting of hexamidine, thymol, polyvinyl formate, niacinamide, cinnamon essential oil, cinnamon bark essential oil, cinnamic aldehyde, piroctone olamine, octenidine dihydrochloride, polyquaternium, and combinations thereof.
X. The deodorant composition according to paragraphs A to W, wherein the refractive index of the polar phase is from 1.3500 to 1.4300.
Y. A deodorant composition, said composition comprising:
   a primary carboxylic acid, wherein the primary carboxylic acid has a C Log D less than −0.5 at a pH from 3.0 to 5.0;
   wherein the deodorant composition is a polar in nonpolar emulsion, comprising a polar phase, a nonpolar phase and an emulsifier phase;
   wherein the polar phase has a refractive index that is at most 0.01 different than the nonpolar phase;
   wherein the composition is free of aluminum;
   wherein the primary carboxylic acid has a surface tension that is at least 69.8 mN/m when measured in a 2 wt % mixture in water.
Z. A deodorant composition, said composition comprising:
   a primary carboxylic acid, wherein the primary carboxylic acid has a C Log D less than −0.5 at a pH from 3.0 to 5.0;
   wherein the deodorant composition is a polar in nonpolar emulsion, comprising a polar phase, a nonpolar phase and an emulsifier phase;
   wherein the polar phase has a refractive index that is at most 0.01 different than the nonpolar phase;
   wherein the composition is free of aluminum;
wherein the primary carboxylic acid has a Log S greater than 2.5 mol/L at a pH from 3.0 to 5.0 at 25° C.
AA. A deodorant composition comprising:
   from about 2% to about 15%, preferably from about 2.5% to about 12%, more preferably from about 3% to about 10%, and even more preferably from about 4% to 8% of one or more carboxylic acids comprising:
   one or more carboxylic acids having a C Log D of less than −0.5 at a pH from 3.0 to 5.0; and
   optionally one or more carboxylic acids having a C Log D of greater than 0.5 at a pH from 3.0 to 5.0;
   wherein the weight ratio of the C Log D greater than or equal to −0.5 at a pH from 3.0 to 5.0 to carboxylic acids having a C Log D less than −0.5 at a pH from 3.0 to 5.0 is from 0 to about 2.8:1; preferably from 0 to about 2:1, more preferably 0 to about 1:1, more preferably 0 to about 0.5:1, more preferably 0 to about 0.25:1, more preferably 0 to about 0.1:1, more preferably from 0 to about 0.05:1;
from about 15% to about 45% water, preferably from about 20% to about 45% water, more preferably from about 30% to about 45% water;
wherein the deodorant composition is a polar in nonpolar emulsion, comprising a polar phase, a nonpolar phase and an emulsifier phase;
wherein the composition is free of aluminum.
BB. The deodorant composition according to Paragraphs A-AA, wherein the composition is substantially free of or free of a carboxylic acid having a C Log D of greater than 0.5 at a pH from 3.0 to 5.0.
CC. Use of a deodorant composition according Paragraphs A-BB, for reducing underarm irritation.
DD. Use of a deodorant composition according Paragraphs A-BB, for preventing underarm irritation.
EE. Use of a deodorant composition according to according to Paragraphs A-BB, for providing wetness protection.
FF. Use of a deodorant composition comprising the carboxylic acid according to Paragraphs A-BB, for providing wetness protection.
GG. Use of a deodorant composition comprising the carboxylic acid according to Paragraphs A-FF, for providing underarm malodor control.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”. All numeric values (e.g., dimensions, flow rates, pressures, concentrations, etc.) recited herein may be modified by the term “about”, even if not expressly so stated with the numeric value.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A deodorant composition comprising:
a primary carboxylic acid, wherein the primary carboxylic acid has a C Log D less than −0.5 at a pH from 3.0 to 5.0;
wherein the deodorant composition is a polar in nonpolar emulsion, comprising a polar phase, a nonpolar phase and an emulsifier phase;
wherein the polar phase has a refractive index that is at most 0.01 different than the nonpolar phase;
wherein the composition is free of aluminum;
wherein the primary carboxylic acid has a surface tension that is at least 69.8 mN/m when measured in a 2 wt % mixture in water.

2. The deodorant composition of claim 1, wherein the polar phase has a surface tension that is at least 20.0 mN/m greater than the nonpolar phase.

3. The deodorant composition of claim 1, wherein the primary carboxylic acid has a Log S greater than 2.5 mol/L at a pH from 3.0 to 5.0 at 25° C.

4. The deodorant composition of claim 1, wherein the primary carboxylic acid has a C Log D less than −0.5 at a pH from 3.0 to 4.5.

5. The deodorant composition of claim 1, wherein the primary carboxylic acid is chosen from lactic acid, malic acid, tartaric acid, succinic acid, vitamin C, tetrahydrofuran dicarboxylic acid, glycolic acid, oxalic acid, gluconic acid, salts thereof, or mixtures thereof.

6. The deodorant composition of claim 1, wherein the polar in nonpolar emulsion is a polar in silicone emulsion.

7. The deodorant composition of claim 1, wherein the polar in nonpolar emulsion is a water in oil emulsion.

8. The deodorant composition of claim 1, wherein the polar in nonpolar emulsion is a water in silicone emulsion.

9. The deodorant composition of claim 1, wherein the polar phase comprises at least about 75% of the composition.

10. The deodorant composition of claim 1, wherein the primary carboxylic acid has a C Log D less than −0.6 at a pH from 3.0 to 5.0.

11. The deodorant composition of claim 1, wherein the polar phase has a surface tension that is at least 21 mN/m greater than the surface tension of the nonpolar phase.

12. The deodorant composition of claim 1, wherein the polar phase has a refractive index that is at most 0.005 different than the polar phase.

13. The deodorant composition of claim 1, wherein the composition has a percent transmittance (% T) of at least about 80% transmittance at 600 nm.

14. The deodorant composition of claim 1, wherein the composition further comprises glycerin.

15. The deodorant composition of claim 1, wherein the composition further comprises PEG.

16. The deodorant composition of claim 1, further comprising an antimicrobial chosen from hexamidine, thymol, polyvinyl formate, niacinamide, cinnamon essential oil, cinnamon bark essential oil, cinnamic aldehyde, piroctone olamine, octenidine dihydrochloride, polyquaternium, or mixtures thereof.

17. The deodorant composition of claim 1, wherein the refractive index of the polar phase is from 1.3500 to 1.4300.

18. A deodorant composition comprising:
from about 2% to about 15% of one or more carboxylic acids comprising:
one or more carboxylic acids having a C Log D of less than −0.5 at a pH from 3.0 to 5.0; and
optionally one or more carboxylic acids having a C Log D of greater than or equal to −0.5 at a pH from 3.0 to 5.0;
wherein the weight ratio of the C Log D greater than or equal to −0.5 at a pH from 3.0 to 5.0 to carboxylic acids having a C Log D less than −0.5 at a pH from 3.0 to 5.0 is from from 0 to about 2.8:1;
from about 15% to about 45% water;
wherein the deodorant composition is a polar in nonpolar emulsion, comprising a polar phase, a nonpolar phase and an emulsifier phase;
wherein the composition is free of aluminum.

19. The deodorant composition of claim 18, wherein the carboxylic acid has a surface tension that is at least 69.8 mN/m when measured in a 2 wt % mixture in water.

20. A deodorant composition, said composition comprising:
a primary carboxylic acid, wherein the primary carboxylic acid has a C Log D less than −0.5 at a pH from 3.0 to 5.0;
wherein the deodorant composition is a polar in nonpolar emulsion, comprising a polar phase, a nonpolar phase and an emulsifier phase;
wherein the polar phase has a refractive index that is at most 0.01 different than the nonpolar phase;
wherein the composition is free of aluminum;
wherein the primary carboxylic acid has a Log S greater than 2.5 mol/L at a pH from 3.0 to 5.0 at 25° C.
